(19) United States
(12) Patent Application Publication
(10) Pub. No.: US 2006/0100713 A1
(43) Pub. Date: May 11, 2006

(54) Mobile bearing total elbow prosthesis, ulnar component, and associated kit

(76) Inventor: Robert J. Ball, San Marcos, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/256,010

(22) Filed: Oct. 21, 2005

Related U.S. Application Data
(60) Provisional application No. 60/623,372, filed on Oct. 29, 2004.
(60) Provisional application No. 60/623,195, filed on Oct. 29, 2004.

(51) Int. Cl. A61F 2/38

(52) U.S. Cl. 623/20.13; 623/20.11

(57) ABSTRACT

An elbow prosthesis is provided. The elbow prosthesis includes an ulnar component. The ulnar component includes a first portion implantable in a cavity formed in the ulna and a second portion. The first portion of the ulnar component defines a longitudinal axis. The second portion of the ulnar component is rotatably connected to the first portion of the ulnar component about an axis normal to the longitudinal axis. The elbow prosthesis also includes a humeral component including a first portion implantable in a cavity formed in the humerus and a second portion. The second portion of the humeral component is operably connected to the second portion of the ulnar component.

### TECHNICAL FIELD OF THE INVENTION

The present invention relates generally to the field of orthopaedics, and more particularly, to artificial joints and, in particular, to a modular elbow prosthesis.

### CROSS-REFERENCE TO RELATED APPLICATIONS

This Application is a Utility Application based upon U.S. Provisional Patent Application Ser. No. 60/623,372 filed Oct. 29, 2004, entitled “MODULAR TOTAL ELBOW PROSTHESIS & INSTRUMENTS AND ASSOCIATED METHOD and upon U.S. Provisional Patent Application Ser. No. 60/623,195 filed Oct. 29, 2004, entitled “MOBILE BEARING TOTAL ELBOW PROSTHESIS & INSTRUMENTS AND ASSOCIATED METHOD”. Cross reference is made to the following applications: U.S. Provisional Patent Application Ser. No. 60/623,372 filed Oct. 29, 2004, entitled “MODULAR ELBOW PROSTHESIS & INSTRUMENTS AND ASSOCIATED METHOD”, U.S. Provisional Patent Application Ser. No. 60/623,195 filed Oct. 29, 2004, entitled “MOBILE BEARING TOTAL ELBOW PROSTHESIS & INSTRUMENTS AND ASSOCIATED METHOD”, as well as DEP645USNP entitled “MODULAR TOTAL ELBOW PROSTHESIS, INSTRUMENTS AND ASSOCIATED METHOD”, DEP645USNP1 entitled “MODULAR TOTAL ELBOW PROSTHESIS, HUMERAL COMPONENT AND ASSOCIATED KIT”, DEP645USNP2 entitled “MODULAR TOTAL ELBOW HUMERAL COMPONENT AND ASSOCIATED METHODS”, and DEP0645USNP3 entitled “MOBILE BEARING TOTAL ELBOW PROSTHESIS, HUMERAL COMPONENT, AND ASSOCIATED METHOD” all filed concurrently herewith and all incorporated herein by reference.

### BACKGROUND OF THE INVENTION

A joint within the human body forms a juncture between two or more bones or other skeletal parts. The ankle, hip, knee, shoulder, elbow and wrist are just a few examples of the multitude of joints found within the body. As should be apparent from the above list of examples of joints, many of the joints permit relative motion between the bones. For example, the motion of sliding, gliding, and hinge or ball and socket movements may be had by a joint. For example, the ankle permits a hinge movement, the knee allows for a combination of gliding and hinge movements and the shoulder and hip permit movement through a ball and socket arrangement.

The joints in the body are stressed or can be damaged in a variety of ways. For example, gradual wear and tear is imposed on the joints through the continuous use of a joint over the years. The joints that permit motion have cartilage positioned between the bones providing lubrication to the motion and also absorbing some of the forces direct to the joint. Over time, the normal use of a joint may wear down the cartilage and bring the moving bones in direct contact with each other. In contrast, in normal use, a trauma to a joint, such as the delivery of a large force from an accident, for example an automobile accident, may cause considerable damage to the bones, the cartilage or to other connective tissue such as tendons or ligaments.

Arthropathy, a term referring to a disease of the joint, is another way in which a joint may become damaged. Perhaps the best known joint disease is arthritis, which is generally referred to as a disease or inflammation of a joint that results in pain, swelling, stiffness, instability, and often deformity.

There are many different forms of arthritis, with osteoarthritis being the most common and resulting from the wear and tear of the cartilage within a joint. Another type of arthritis is osteonecrosis, which is caused by the death of a part of the bone due to loss of blood supply. Other types of arthritis are caused by trauma to the joint while others, such as rheumatoid arthritis, Lupus, and psoriatic arthritis destroy cartilage and are associated with the inflammation of the joint lining. In the human elbow, three degrees of freedom are present. These are flexion-extension, varus-valgus (carrying angle) and prunation/supination.

Various elbow prosthesis have been constructed as a replacement for the natural human elbow. The two basic types of elbow prosthesis known in the prior art are semi-constrained and unconstrained. In semi-constrained prosthesis, the prosthetic joint is held together mechanically, by components of the prosthesis. Such devices are shown, for example, in U.S. Pat. No. 5,376,121 to Huene et al., U.S. Pat. No. 3,708,805 to Scales, et al., U.S. Pat. No. 3,939,496 to Ling, et al., and U.S. Pat. No. 4,224,695 to Grundei, et al. In an unconstrained device, the prosthetic device is held together by the patient's natural soft tissues. Such a device is shown in U.S. Pat. No. 4,293,963 to Gold, et al. In each of these devices, one portion of the prosthesis is implanted in the humerus of the patient and the other portion is implanted in the ulna. The two portions then mate in some manner to allow articulation of the joint. In the '695 patent to Grundei, et al., an additional portion of the prosthesis is implanted in the radius of the patient.

A surgeon may not always know prior to beginning an operation whether a patient would be better served by a semi-constrained or unconstrained elbow prosthesis. Thus, it would be desirable to provide an elbow prosthesis that may be utilized in either the semi-constrained or unconstrained manner.

It may also be necessary to convert an unconstrained elbow prosthesis to a semi-constrained one, or vice versa, after implantation and use for a period of time. In order to do so, it is typically necessary to remove the portion of the prosthesis implanted in the humerus and ulna and to replace the entire prosthesis with either the semi-constrained or unconstrained variety.

Prosthetic elbows currently marketed typically can be implanted to operate in one of two ways. The first way is in an unconstrained or also known as unlinked manner. The other way that currently market elbows can operate is as a semi-constrained or a linked prosthesis. Unconstrained prosthetic elbows are more generally indicated for osteoarthritic or post-traumatic patients with strong soft tissue about the elbow, while the joint surfaces are arthritic and painful. Unconstrained elbows typically make use of a metal humeral articulating surface and a polyethylene ulna-articulating surface. Each of the articulating surfaces has matching either convex or concave surfaces, respectively. Semi-constrained prosthesis are used in patients with inflammatory disease, which results in weaker soft tissues and bone erosion. This type of prosthesis uses a linkage pin at the elbow axis of rotation.

The Acclaim elbow manufactured by DePuy Orthopaedics, Inc., can currently be converted from unconstrained to semi-constrained interoperatively. The Acclaim elbow is more fully described in U.S. Pat. No. 6,027,534 and U.S. Pat. No. 6,290,725 incorporated herein in their entireties by reference.

An elbow prosthesis can be further advanced by further modular features in anatomic considerations. These features can reduce manufacturing, allow closer match to patient anatomy, and make the prosthesis easier to implant.

While current convertible prosthesis have been accepted in the market place, two problems exist which are inherent to the design of the prosthesis, and which if solved would enhance the manufacturing, surgical technique, and performance of the design. First, the configuration of all semi-constrained implants makes use of an axis pin mechanism for preventing dislocation and positioning of axis of articulation. Implants currently on the market require substantial amounts of condylar bone to be removed if the polyethylene wears and needs replacement.

As with other orthopaedic devices, it is arguable that long term successes of the device is at least partially dependent on the fit of the prosthesis to the patient. Currently there are no devices that are marketed for the elbow that allow the surgeon to fit the stem to the canal and independently fit the head shape to the condylar area.

Manufacturing costs of current elbow prosthesis are also a consideration. High costs are associated with the manufacturing of the connecting feature, for example, Morse taper features on the stem and condylar portion of the prosthesis.

The Stryker Howmedica Osteonics elbow prosthesis manufactured and sold as the Solar™ elbow prosthesis has a described technique of drilling holes in the condyles for removal of the axis assembly. Such extra drilling or removing of condylar material from the bone is not optimal as the holes must be of sufficient diameter to severely weaken the supercondylar regions of the humerus. This weakening of the humerus lessens the value of this design feature.

Three very critical pivoting abilities on degrees of freedom occur in the natural elbow. One of these degrees of freedom is known as Peg Shift Carrying Angle. This degree of freedom permits the hand to carry, for example, a bucket at various orientations.

The second of these motions or degrees of freedom is known as Peg Shift Version and represents the ability of the humerus to rotate along the longitudinal axis of an extended arm with respect to the ulna. This motion permits the carrier to, for example, rotate the handle of a bucket in a carrying position.

The third of these motions is the gross rotation between the humerus and ulna/radius, flexion and extension.

### SUMMARY OF THE INVENTION

According to the present invention, the pin axis of the elbow prosthesis of the present invention is modular and provides a junction that is further proximal in the humeral component. A set of stems may be used and designed to fit the patient's anatomy for indications that would be available to fit with several types of articulating components. The articulating components may have varying anatomic features to match patient anatomy as well as to offer the ability to convert to an unconstrained to a semi-constrained application.

In addition, the articulating surfaces of the humerus may be modified to include the use of a radial head prosthesis. In other words, the humeral component may be modular and provide for rotation between the components about the longitudinal axis of the humerus. The modularity of the design of the present invention is such that the humeral articulating head for an unconstrained prosthesis can be removed and replaced by a yoke type device for a semi-constrained prosthesis without removal of additional bone or soft tissue.

The modular junction of the present invention, allows the implant to be converted from an unconstrained to a semi-constrained prosthesis. The modular junction is moved proximal in the bone so that the removal of the polypin axial system is more proximal. The new modular junction of the present invention between the stem and articulating head allows one to customize the stem shape and size to the patient's anatomy and also allows the bearing mechanism to be assembled after cementing of the prosthetic stem.

The stem may have a tapered post concentric about the stem longitudinal axis and extend distally. The tapered stem may fit securely within a tapered hole within the unconstrained or semi-constrained bearing surface head. Alternatively, and according to the present invention, the post may fit rotatably in a hole in the unconstrained or semi-constrained bearing surfaces to permit a mobile bearing or rotation of the components of the humeral component. A wide range of embodiments of the present invention, may include the reversal of the taper of the assembly mechanism, further modularity separating the stem, body, and head. Dual or square taper or other configurations may be used.

The modularity of the design of the present invention allows many options combining specially designed components to create a prosthesis that more accurately fits the patient's need. A three-part design for the prosthesis may be provided providing a stem component, a condylar component, and an articulating component. For example, the ulnar component and the humeral component, or only one of them, may have a three-part construction. The modularity also occurs in the portion incorporating the mobile bearing concept to the design. One way to provide for a mobile bearing configuration is to allow the junction between the stem and the head to be loose, providing translation and rotation around the junction.

According to one embodiment of the present invention, there is provided an elbow prosthesis. The elbow prosthesis includes an ulnar component. The ulnar component has a first portion of the ulnar component that is implantable in a cavity formed in the ulna. The ulnar component has a second portion operably associated with the first portion. The elbow prosthesis also includes a humeral component having a first portion of the humeral component that is implantable in a cavity formed in the humerus. The first portion of the humeral component defines a longitudinal axis of the first portion and a second portion of the humeral component. The humeral component also has a second portion operably connected to the second portion of the ulnar component. The second portion of the humeral component is rotatably connected to the first portion of the humeral component about the longitudinal axis.

According to another embodiment of the present invention, there is provided an elbow prosthesis. The elbow prosthesis includes an ulnar component including a first portion of the ulnar component implantable in a cavity formed in the ulna and a second portion of the ulnar component. The first portion of the ulnar component defines a longitudinal axis of the ulnar component. The second portion of the ulnar component is rotatably connected to the first portion of the ulnar component about an axis normal to the longitudinal axis. The elbow prosthesis also includes a humeral component including a first portion of the humeral component. The first portion is implantable in a cavity formed in the humerus. The humeral component also includes a second portion operably connected to the first portion of the humeral component.

According to another embodiment of the present invention there is provided a humeral component for use with an ulnar component to form an elbow prosthesis. The humeral component includes a first portion thereof implantable in a cavity formed in the humerus. The first portion defines a longitudinal axis of the first portion thereof. The humeral component also includes a second portion thereof. The second portion is adapted to be operably connected to the ulnar component. The second portion is rotatably connected to the first portion about the longitudinal axis.

According to yet another embodiment of the present invention there is provided an ulnar component for use with a humeral component to form an elbow prosthesis. The ulnar component includes a first portion implantable in a cavity formed in the ulna. The first portion defines a longitudinal axis of the first portion. The ulnar component also includes a second portion. The second portion is rotatably connected to the first portion about an axis normal to the longitudinal axis.

According to still another embodiment of the present invention there is provided a kit for use in performing total elbow arthroplasty. The kit includes an ulnar stem component for implantation at least partially in the ulnar medullary canal and an ulnar hinge component attachable to the ulnar stem component. The kit also includes a humeral stem component for implantation at least partially into the humeral medullary canal. The humeral stem component defines a longitudinal axis thereof. The kit also includes a first humeral hinge component removably attachable to the humeral stem component and rotatable with respect to the humeral stem component about the longitudinal axis. The kit also includes a second humeral hinge component removably attachable to the humeral stem component and rotatable with respect to the humeral stem component about the longitudinal axis. The second humeral hinge component has at least one dimension different than the first humeral hinge component.

According to still another embodiment of the present invention there is provided a kit for use in performing total elbow arthroplasty. The kit includes an ulnar stem component for implantation at least partially into the ulnar medullary canal. The ulnar stem component defines a longitudinal axis thereof. The kit also includes a first ulnar hinge component attachable to the ulnar stem component for rotatable operation therewith. The second ulnar hinge component being different in at least one of size and shape than the first ulnar hinge component. The kit also includes a second ulnar hinge component attachable to the ulnar stem component for rotatable operation therewith about an axis normal to the longitudinal axis of the ulnar stem component. The kit also includes a humeral stem component for implantation at least partially in the humeral medullary canal. The humeral stem component defines a longitudinal axis thereof. The kit also includes a humeral hinge component attachable to the humeral stem component and adapted for cooperation with one of the ulnar hinge component.

According to a further embodiment of the present invention, there is provided a method for providing total elbow arthroplasty. The method includes the steps of providing a elbow prosthesis kit including an ulnar stem component, an ulnar hinge component, a humeral stem component, a fixed bearing humeral hinge component, and a mobile bearing humeral hinge component, cutting an incision in the patient, preparing the humeral and ulnar cavity, assembling the chosen of a fixed bearing humeral hinge component and a mobile bearing humeral hinge component onto the humeral stem component, and implanting the humeral stem component in the humeral cavity.

The technical advantages of the present invention, include the ability to provide for a mobile bearing configuration in an elbow prosthesis. For example, according to one aspect of the present invention, an elbow prosthesis is provided including an ulnar component and a humeral component. The humeral component includes a first portion for implantation in a cavity. The first portion defines a longitudinal axis of the humeral component. The humeral component further includes a second portion rotatively connected to the first portion of the humeral component about the longitudinal axis. Thus, the present invention provides for the ability to provide for a mobile bearing configuration.

The technical advantages of the present invention include the ability to provide for a mobile bearing configuration of an elbow with the mobile bearing feature being about the pronation/supination axis. For example, according to another aspect of the present invention, an elbow prosthesis is provided including an ulnar and a humeral component. The ulnar and humeral components operatively cooperate with each other. The ulnar component includes a first portion implantable in a cavity and defining a longitudinal axis thereof. The ulnar component further includes a second portion being rotatively connected to the first portion of the ulnar component about axis normal to the longitudinal axis. Thus, the present invention provides a mobile bearing configuration for rotation normal to the ulna to provide for a prosthesis that rotates about the peg shift-carrying angle.

The technical advantages of the present invention, further include the ability to reduce condylar bone removal if the polyethylene peg axis is repaired. For example, according to yet another aspect of the present invention, an elbow prosthesis is provided with an ulnar component and a humeral component. The rotational axis of the humeral component is provided with a proximal position. Thus, the present invention provides for reduced condylar bone removal when the poly/pin axis is repaired.

The technical advantage of the present invention also includes the ability to fit the stem shape to the canal and to fit the head shape to the condylar area. For example, according to another aspect of the present invention, either the ulnar or humeral component of the elbow prosthesis, or both, are modular or include two separatable pieces—one fitted into the canal and the other to the condylar area. Therefore, separate stems and articulating portions can be provided to have the prosthesis better fit the condylar area.

The technical advantages of the present invention further include the ability to lower manufacturing costs and to provide a less expensive tapered junction. For example, according to yet another aspect of the present invention, an elbow prosthesis is provided with a multi-piece humeral or ulnar component, which includes a tapered junction. One of the portions of the component has a conifrustrical-tapered component and the other has a conifrustrical cavity to receive the protrusion. Thus, the present invention provides for lower manufacturing cost by providing a less expensive junction, compared to multicomponent screw mechanisms or squared tapered junctions.

The technical advantages of the present invention further include the ability to convert the prosthesis from an unconstrained prosthesis to a semi-constrained prosthesis without removal of soft tissue or bone. For example, and according to another aspect of the present invention, an ulnar or a humeral component is provided with a first portion for implantation in a cavity and a second portion connected to the first portion positioned in the condylar area of the bone. The second portion may be connected to the first portion along the longitudinal axis of the component to permit the conversion of an unconstrained to a semi-constrained prosthesis without removing soft tissue or bone.

The technical advantages of the present invention also include the ability to more closely fit the prosthetic features to individual patient anatomy including the patient bone stem size, the patient bone bearing surface sizes, and the location of the joint or the bearing mechanism for both semi-constrained and unconstrained prosthesis. For example, and according to yet another aspect of the present invention, an elbow prosthesis is provided with an ulnar component and a humeral component. The ulnar component or the humeral component, or both, include a first component for positioning in the stem and a second component for positioning in the condylar area. By providing the modularity or multiple piece construction for the humeral and ulnar components, a series of ulnar and humeral components may be mixed or matched to provide a close match to the anatomical features of individual patients.

The technical advantages of the present invention, include the ability to provide a more dimensional tolerant design. For example, and according to yet another aspect of the present invention, a modular long bone component of an elbow prosthesis is provided with a tapered junction with adjacent opposing faces on the components. The opposing faces can be used to control the location of the axis hole, to provide a position that is not affected by the tapered surface accuracy of the tapered junction.

The technical advantages of the present invention further include the ability to provide for optimal materials and coatings as well as surface treatments to the components. For example, according to yet another aspect of the present invention, an elbow prosthesis is provided with a long bone component that is modular including stem and condylar portions. The stem or condylar portions may be coated for bone growth, etc. or each may have its own individual surface treatment, therefore allowing optimal material surfaces and coating treatments.

The technical advantages of the present invention also include the ability to permit easier surgical techniques that can select the type, size, and position of articulating surfaces after cementing the stem. For example, and according to yet another aspect of the present invention, a surgical technique is provided whereby an elbow prosthesis is provided separate stem and condylar portions for the ulnar and humeral components of the elbow prosthesis. The stem components are positioned in the bone and later the condylar portions are secured to the cemented stems. The condylar portions may be interchangeably selected and assembled to the cemented stems. Thus the present invention provides for an easier surgical technique to select the size and type of position of the articulating surface after cementing the stems. An aspect of the present invention provides for being able to select between semi-constrained and non-constrained elbow configurations after cementing the ulnar stem and the humeral stem.

Other technical advantages of the present invention will be readily apparent to one skilled in the art from the following figures, descriptions and claims.

### BRIEF DESCRIPTION OF THE DRAWINGS

For a more complete understanding of the present invention and the advantages thereof, reference is now made to the following description taken in connection with the accompanying drawings, in which:

FIG. 1 is an exploded anterior/posterior view of a semi-constrained elbow prosthesis including the capability of rotation about peg shift version in accordance with an embodiment of the present invention;

FIG. 1A is a partial plan view, partially in cross-section of another embodiment of the present invention showing an alternate connection construction;

FIG. 1B is a partial plan view, partially in cross-section of another embodiment of the present invention showing an alternate connection construction;

FIG. 1C is a partial plan view, partially in cross-section of another embodiment of the present invention showing an alternate connection construction;

FIG. 1D is a plan view of a humeral prosthesis in accordance with yet another embodiment of the present invention;

FIG. 1E is a plan view of an ulnar component in accordance with another embodiment of the present invention;

FIG. 2 is a plan view of a humeral component of the elbow prosthesis of FIG. 1 showing rotation of the prosthesis about peg shift long axis carry angle;

FIG. 3 is an anterior/posterior view of a humerus, ulna and radius of a patient showing the various available motions of the patient utilizing an elbow prosthesis in accordance with various embodiments of the present invention;

FIG. 4 is a perspective view of the humeral stem component of the prosthesis of FIG. 1;

FIG. 5 is a perspective view of the humeral articulating component;

FIG. 6 is an exploded perspective view of the semi-constrained humeral articulating component of FIG. 5 that may be installed on the humeral stem component of FIG. 4 to form the semi-constrained humeral assembly;

FIG. 7 is an exploded plan view of the pin assembly of the semi-constrained elbow prosthesis of FIG. 1;

FIG. 8 is a partial plan view of the pin assembly of FIG. 7 installed into the semi-constrained elbow prosthesis of FIG. 1;

FIG. 9 is an exploded anterior/posterior view of a semi-constrained elbow prosthesis including the capability of rotation about long axis including a bearing positioned between the humeral stem component and the humeral articulating component in accordance with another embodiment of the present invention;

FIG. 10 is an exploded perspective view of the semi-constrained humeral assembly of the semi-constrained elbow prosthesis of FIG. 9;

FIG. 10A is an exploded anterior/posterior view of an unconstrained elbow prosthesis corresponding to the semi-constrained elbow prosthesis of FIG. 9;

FIG. 11 is a plan view of a semi-constrained elbow prosthesis with rotation of the carrying angle in accordance with another embodiment of the present invention;

FIG. 12 is an exploded anterior/posterior view of a semi-constrained elbow prosthesis including the capability of rotation in the carrying angle in accordance with yet another embodiment of the present invention;

FIG. 12A is an exploded anterior/posterior view of an unconstrained elbow prosthesis corresponding to the semi-constrained elbow prosthesis of FIG. 12;

FIG. 13 is a plan view of the semi-constrained humeral stem of the semi-constrained elbow prosthesis of FIG. 12 in position on the humerus;

FIG. 14 is an enlarged plan view of the semi-constrained humeral stem of FIG. 13;

FIG. 15 is a plan view of the ulnar stem component of the ulnar assembly of the semi-constrained elbow prosthesis of FIG. 12;

FIG. 16 is a side view of the ulnar stem component FIG. 15;

FIG. 17 is a perspective view of the ulnar stem component FIG. 15;

FIG. 18 is a plan view of the ulnar articulating component of the ulnar assembly of the semi-constrained elbow prosthesis of FIG. 12;

FIG. 19 is a side view of the ulnar articulating component FIG. 18;

FIG. 20 is a perspective view of the ulnar articulating component FIG. 18;

FIG. 21 is a plan view of the semi-constrained ulnar articulating component of FIG. 18 installed on the ulnar stem component of FIG. 15 to form the semi-constrained ulnar assembly;

FIG. 21A is a plan view of another embodiment of the present invention in the form of a semi-constrained ulnar articulating component similar to that of FIG. 21 show in posterior and against stops of an ulnar stem component similar to that of FIG. 21 having stops to restrict the rotation of the articulating component;

FIG. 22 is a plan view of an alternate design of a semi-constrained ulnar assembly in accordance with yet another embodiment of the present invention for use with the humeral assembly of the semi-constrained elbow prosthesis of FIG. 12;

FIG. 23 is an exploded anterior/posterior view of a semi-constrained elbow prosthesis including the capability of rotation about long axis version including a bayonet connection between the humeral stem component and the humeral articulating component in accordance with another embodiment of the present invention;

FIG. 24 is a perspective view of the humeral stem component of the semi-constrained elbow prosthesis of FIG. 23;

FIG. 25 is a plan view of the humeral stem component of FIG. 24;

FIG. 26 is a plan view of the semi-constrained humeral articulating component of the semi-constrained elbow prosthesis of FIG. 24 with a portion of the stem component of FIG. 25;

FIG. 26A is a cross sectional view of FIG. 26 along the line 26A-26A in the direction of arrows with the stem component 90° out of location;

FIG. 27 is a side view of the semi-constrained ulnar component of the semi-constrained elbow prosthesis of FIG. 24;

FIG. 28 is an exploded anterior/posterior view of an unconstrained elbow prosthesis including the capability of rotation about peg shift version including a bayonet connection between the humeral stem component and the humeral articulating component in accordance with another embodiment of the present invention;

FIG. 29 is a side view of the humeral articulating component of the unconstrained elbow prosthesis of FIG. 28;

FIG. 30 is a plan view of the ulnar component of the unconstrained elbow prosthesis of FIG. 28;

FIG. 30A is a plan view of another embodiment of the present invention in the form of an ulnar component used for the semi-constrained and unconstrained elbow prosthesis;

FIG. 31 is an exploded anterior/posterior view of an semi-constrained elbow prosthesis including the capability of rotation of the prosthesis in the carry angle as well as including the capability of rotation about long axis including a bearing between the humeral stem component and the humeral articulating component in accordance with yet another embodiment of the present invention;

FIG. 31A is an exploded anterior/posterior view of a semi-constrained humeral assembly for a semi-constrained elbow prosthesis in accordance to another embodiment of the present invention for use with the elbow prosthesis of FIG. 31;

FIG. 32 is an exploded anterior/posterior view of an semi-constrained elbow prosthesis including the capability of rotation of the prosthesis in the carry angle as well as including the capability of rotation about long axis including a bearing between the humeral stem component and the humeral articulating component and further including the capability of conversion to an unconstrained elbow prosthesis in accordance with yet another embodiment of the present invention;

FIG. 32A is an exploded anterior/posterior view of an unconstrained elbow prosthesis corresponding to the semi-constrained elbow prosthesis of FIG. 32;

FIG. 33 is a plan view of a kit for use in total elbow arthroplasty in accordance with another embodiment of the present invention;

FIG. 34 is another plan view of a kit for use in total elbow arthroplasty in accordance with another embodiment of the present invention; and

FIG. 35 is a flow chart of a method for performing total elbow revision arthroplasty in accordance with another embodiment of the present invention.

### DETAILED DESCRIPTION OF THE INVENTION

Embodiments of the present invention and the advantages thereof are best understood by referring to the following descriptions and drawings, wherein like numerals are used for like and corresponding parts of the drawings.

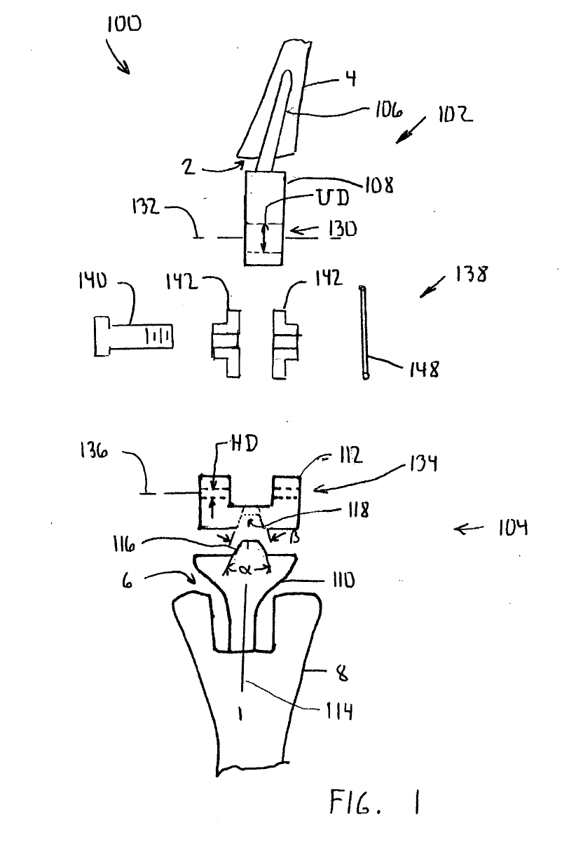

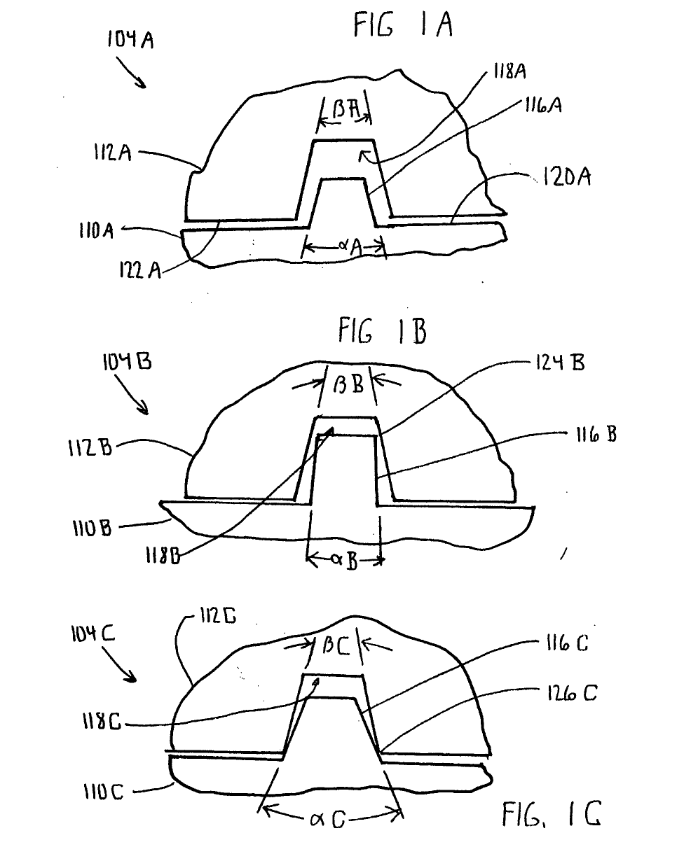

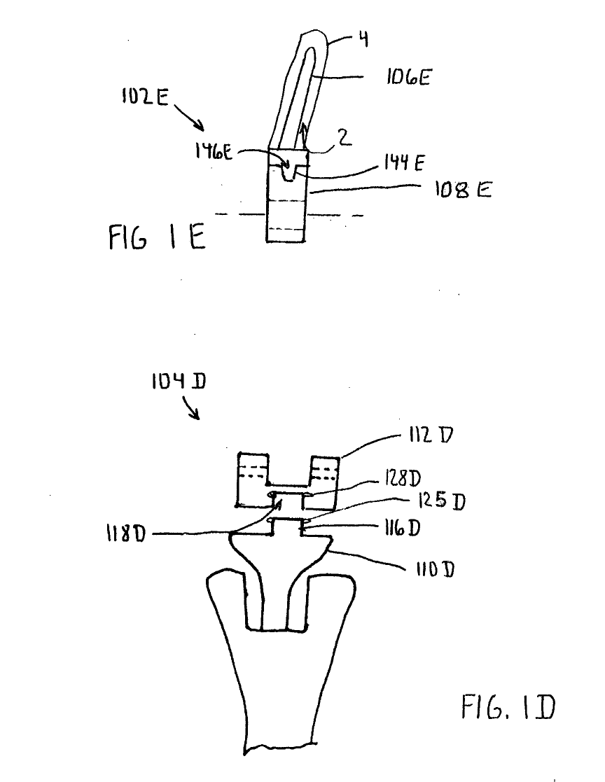

According to the present invention and referring now to FIG. 1, an embodiment of the present invention is shown as elbow prosthesis 100. The elbow prosthesis 100 includes an ulnar component 102 and a humeral component 104. The ulnar component 102 includes a first portion 106 of the ulnar component 102. The first portion 106 is implantable in a cavity 2 formed in the ulna 4. The ulnar component 102 also includes a second portion 108.

The humeral component 104 includes a first portion 110. The first portion 110 is implantable in a cavity 6 formed in the humerus 8. The humeral component 104 also includes a second portion 112 of the humeral component 104. The first portion 110 of the humeral component 104 defines a longitudinal axis 114 of the first portion 110. The second portion 112 of the humeral component is operably connected to the second portion 108 of the ulnar component 102. The second portion 112 of the humeral component 104 is rotatably connected to the first portion 110 of the humeral component 104 about the longitudinal axis 114.

The relative rotational motion of the second portion 112 of the humeral component 104 with respect to the first portion 110 of the humeral component 104 about the longitudinal axis 114 of the humeral component 104 provides for rotation about the long axis of the humerus.

The rotation of the second portion 112 with respect to the first portion 110 may be accomplished in any suitable manner. For example and is shown in FIG. 1, the first portion 110 may include an external protrusion 116 positioned about longitudinal axis 114 of the humeral component 104. The external protrusion 116 cooperates with a cavity 118 formed in the second portion 112. It should be appreciated that the cavity 116 and the protrusion 118 may be reversed with the protrusion being located in the second portion and the cavity in the first portion.

As shown in FIG. 1, the external protrusion may be tapered or have a conofrustical shape. The cavity 118 may likewise have a conifrustical shape for receiving the protrusion 116. In order that the second protrusion 112 may rotate with respect to the first portion 110, preferably, the protrusion 116 of the cavity 118 is adapted to provide for relative motion between each other. (Such relative motion may be accomplished in many ways, for example, by providing the taper angle to be large enough not to provide self-locking.) For example, the included angle α of the first portion 110 may be, for example, a large enough angle i.e. 5°-30° and the included angle β of the cavity 118 of the second portion 112 may likewise have an angle β of 5°-30° so that the second portion 112 does not lock with the first portion 110.

Alternatively, the included angle of the taper in the protrusion 116 and the angle in the cavity 118 may be different to prevent locking of the second portion 112 to the first portion 110.

Alternatively, other designs may be provided to avoid the locking of the second portion to the first portion. For example, and as is shown in FIG. 1A, a humeral component 104A may include a first portion 110A including a protrusion 116A defining an included angle αA. The protrusion 116A mates with cavity 118A formed on second portion 112A. The cavity 118A has an included angle αB. The angles αA and βA are the same.

The first portion 110A of the humeral component 104A includes a first portion face 120A for contact with second portion face 122A of the second portion 112A of the humeral component 104A. The first portion face 120A and the second portion face 122A are adapted to provide for the protrusion 116A to be spaced from the walls of the cavity 118A to provide for motion rotationally of the second portion 112A with respect to the first portion 110A.

Referring now to FIG. 1B, yet another embodiment for providing for first and second portion that may have relative motion between each other is in the form of a humeral component 104B. The humeral component 104B includes a first portion 110B defining a protrusion 116B. The humeral component 104B further includes a second portion 112B defining a cavity 118B. The protrusion 116B and the cavity 118B are provided with different included angles in the respective tapered features. For example, and as is shown in FIG. 1B, the protrusion 116B includes an included angle αB which is smaller than the included angle βB of the cavity 118B. The protrusion 116B contacts the cavity 118B at tip 124B of the protrusion 116B. Thus, the second portion 112 may be rotatable with respect to the first portion 10B.

Referring now to FIG. 1C, yet another embodiment of the present invention to provide for rotation of the second portion with respect to the first portion is shown as humeral component 104C. The humeral component 104C includes a first component 110C defining a protrusion 116C having an included angle αC. The humeral component 104C includes a second component 112C that defines a cavity 118C for cooperation with the protrusion 116C. The cavity 118C defines an included angle βC. The angle βC is greater than the angle αC so that the protrusion 116C contacts the cavity 118C at base 126C of the protrusion 116C. Thus the second portion 112C may rotate with respect to the first portion to 110C.

Referring now to FIG. 1D, yet another embodiment of the present invention is shown as humeral component 104D. The humeral component 104D includes a first portion 110D which is rotatably connected to second portion 112D. The second portion 112D is connected to first portion 110D by means of a cylindrical connection. The first portion 110D includes a cylindrical protrusion 116D which mates with a cylindrical opening 118D formed in the second portion 112D. It should be appreciated that the cylindrical opening 118D may be made for rotatably fitting to cylindrical protrusion 116D. Preferably, the cylindrical opening 118D may be normal to slightly larger than the cylindrical protrusion 116D.

To constrain the second portion 112D in close proximity to the first portion 110D as shown FIG. 1D, the cylindrical protrusion 116D may include a cylindrical rim 125D, which cooperates with groove 128D formed in the second portion 112D of the humeral component 104D. The groove 128D and the rim 125D serve to secure the second portion 112D to the first portion 110D.

Referring again to FIG. 1, the ulnar component 102 is operabley connected to the humeral component 104. As shown in FIG. 1, the elbow prosthesis 100 is a semi-constrained prosthesis. The ulnar component 102 is therefore rotatably connected to the humeral component 104. It should be appreciated that the elbow prosthesis 100 may likewise be designed for use as an unconstrained prosthesis.

While the ulnar component 102 may be operably connected to the humeral component 104 in any suitable manner, for example, and is shown in FIG. 1, the second portion 108 of the ulnar component 104 defines an ulnar opening 130 defining ulnar rotating axis 132. Similarly, the second portion 112 of the humeral component 104 includes a humeral opening 134 defining a humeral rotating axis 136.

A connecting apparatus 138 may be utilized to connect rotatably the ulnar component 102 to the humeral component 104. The connector 138 may have any suitable shape and may as is shown in FIG. 1, include a pin 140. The pin 140 may be directly matingly fitted to 130 and 134 or as shown in FIG. 1, the prosthesis 100 may include a pair of spaced apart bushings 142 which are positioned between the ulnar and humeral components 102 and 104 and fitted to openings 130 and 134.

The components of the elbows prosthesis 100 may be made of any suitable, durable material that is compatible to the human body. For example, the components of the elbow prosthesis 100 may be made of a metal, plastic, or a composite material. If made of a metal, the components of the elbow prosthesis may be made of, for example, cobalt chromium alloy, a stainless steel alloy, or a titanium alloy, if made of a plastic the components of the elbow prosthesis 100 may be made of, for example, polyethylene. If made of polyethylene, the components of the elbow prosthesis 100 may be made of an ultra-high molecular weight polyethylene.

The ulnar component 102 including the first portion 106 and the second portion 108 as well as the humeral component 104 including the first portion 110 and the second portion 112 may be made of a metal. The pin 140 may likewise be made of a metal. The bushings 142 may be made of a plastic or a metal.

As shown in FIG. 1, the ulnar component 102 may be integral or, in other words, the first portion 106 and the second portion 108 may be integral to each other.

Alternatively, according to another embodiment of the present invention and referring now to FIG. 1E, the ulnar component may be in the form of an ulnar component 102E, which is modular. For example, the ulnar component 102E may include a first portion 106E, which is cooperable with the cavity 2 of the ulna 4. The ulnar component 102E may further include a second portion 108E, which is separate from first portion 106E. For example, the first portion 106E may include a protrusion 144E, which cooperates with a cavity 146E formed in the second portion 108E. The protrusion 144E and cavity 146E may be conifrustical or cylindrical or have any suitable shape.

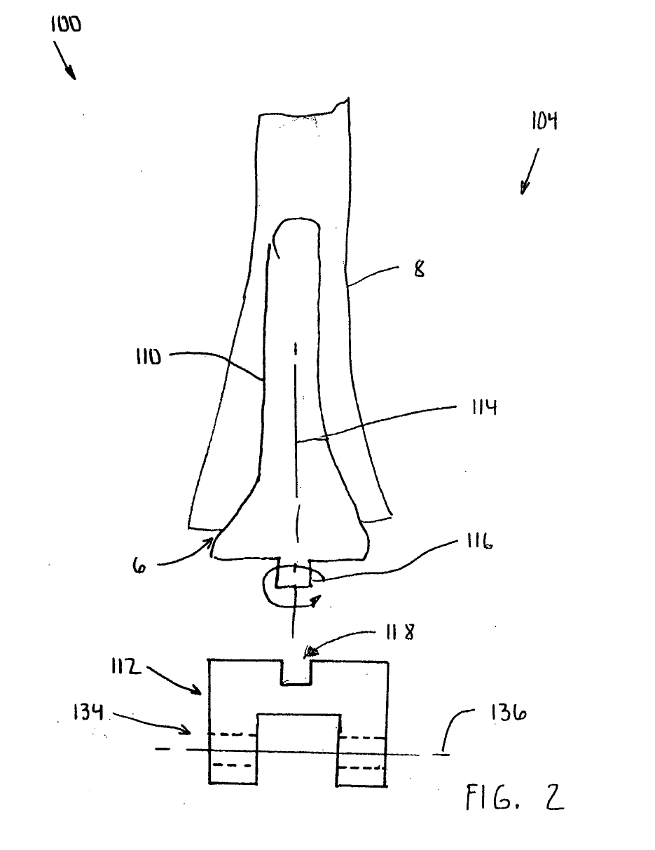

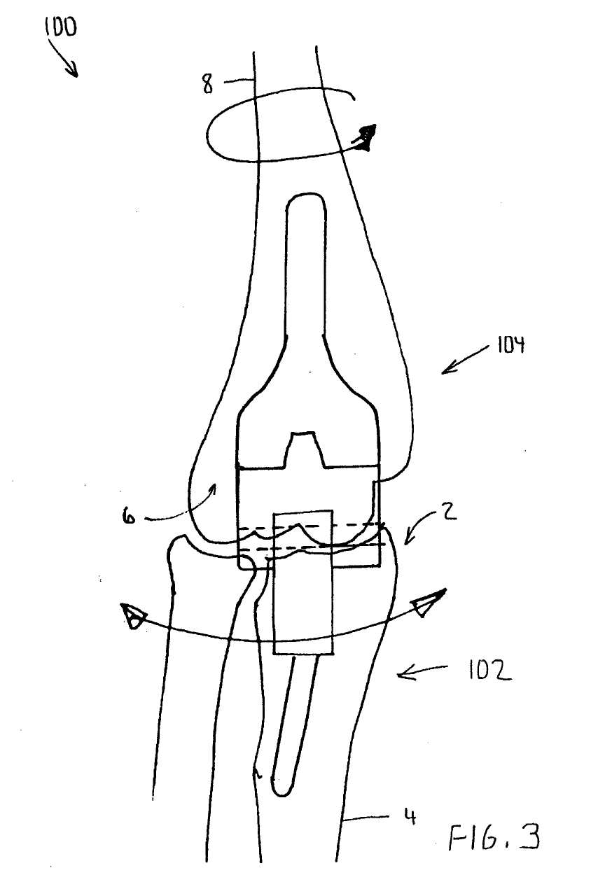

Referring now to FIG. 2, the humeral component 104 of the elbow prosthesis 100 is shown in greater detail. The humeral component 104 includes the first or stem portion 110 which is fitted into the cavity 6 of the humerus 8 as well as the second or articulating portion 112. The articulating portion 112 is connected to the stem portion 110 by means of, for example, the protrusion 116 which extends from the stem portion 110 and which cooperates with the cavity 118 in the articulating portion 112. The articulation portion 112 is permitted to rotate about longitudinal axis 114 of the stem portion 110. The opening 134 is formed in the articulating portion 112 and defines pivoting elbow axis 136.

Referring now to FIG. 3, the elbow prosthesis 100 is shown implanted in the humerus 8 and ulna 4. The elbow prosthesis 100 includes the humeral component 104 which is positioned in the humerus 8 as well as the ulna component 102 which is positioned in the ulna 4.

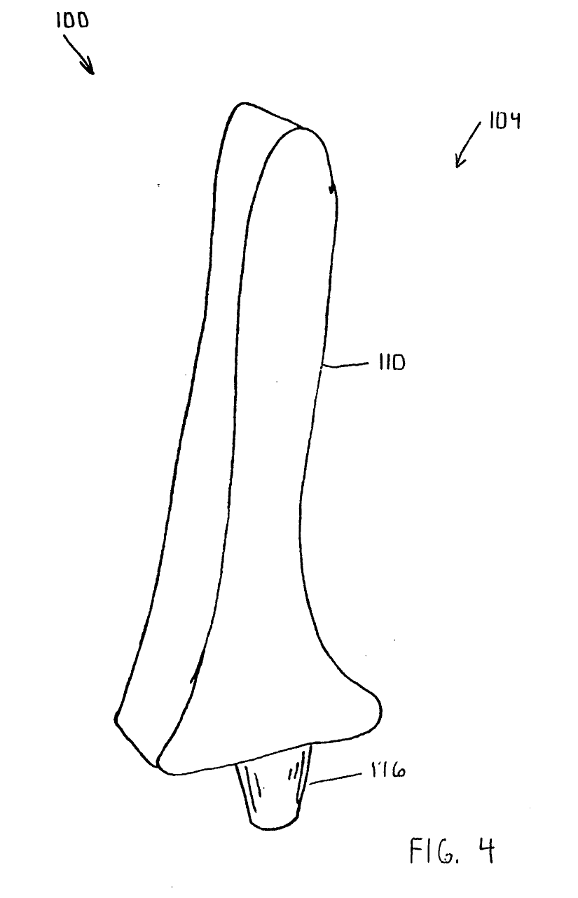

Referring now to FIG. 4, the humeral component 104 of the elbow prosthesis 100 is shown in greater detail. The humeral component 104 includes a stem portion 110 including a conifrustical protrusion 116 extending from an end of the stem portion 110.

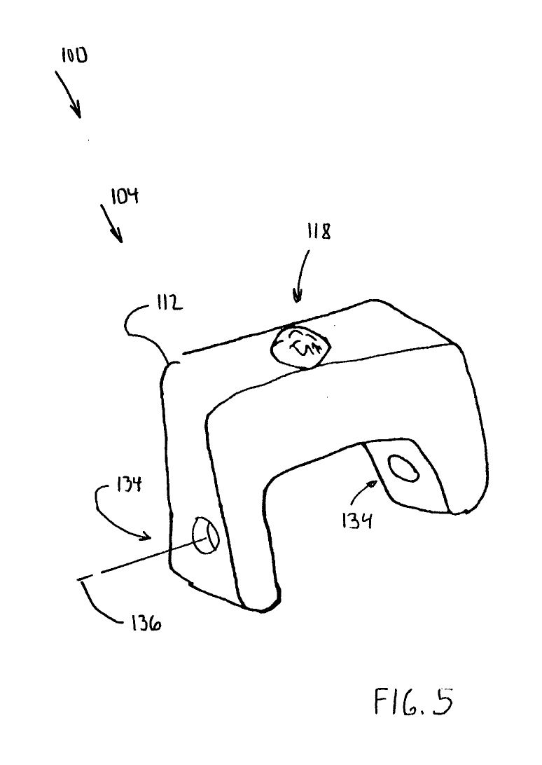

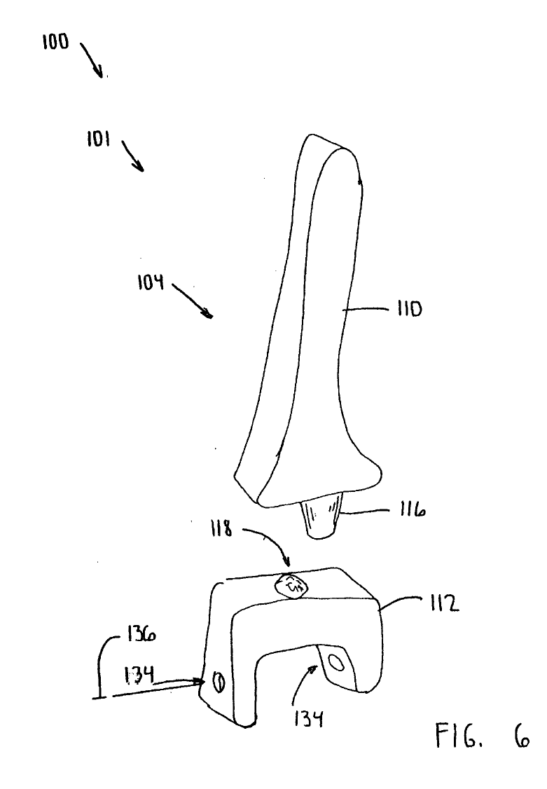

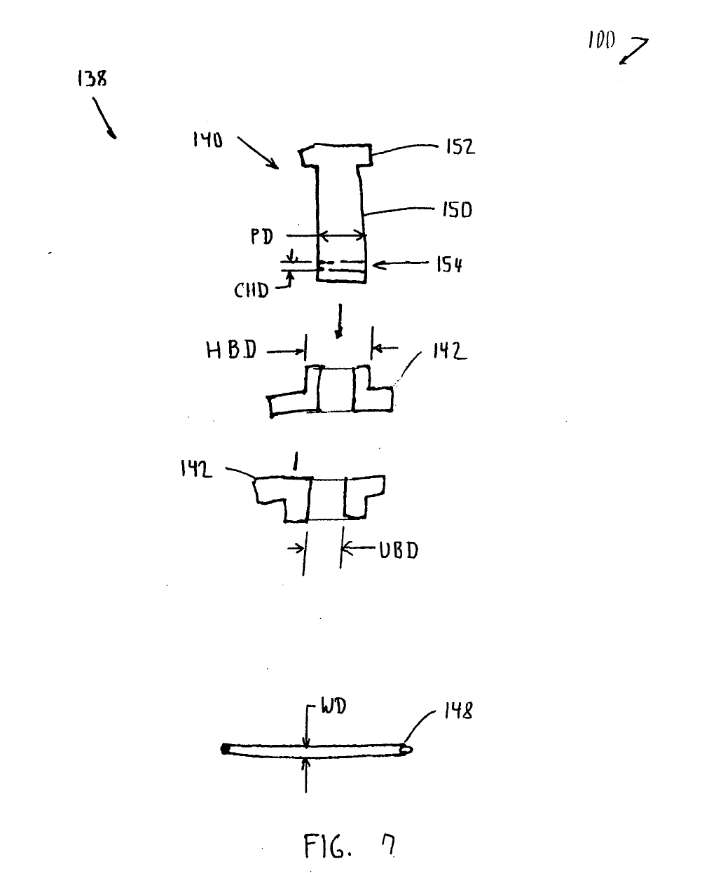

Referring now to FIG. 5, the articulating portion 112 of humeral component 104 of the elbow prosthesis 100 is shown in greater detail. The articulation portion 112 includes the cavity 118 for receiving the protrusion 116 of the stem portion 110 as well as a pair of humeral openings 134 which define rotation axis 136.

Referring now to FIG. 6, the humeral component 104 is shown in a perspective exploded view. The humeral component includes the stem portion 110 which includes the protrusion 116 which cooperates with cavity 118 formed in articulating portion 112 of the humeral component 104. The articulating portion 112 includes the openings 134 defining pivoting center 136.

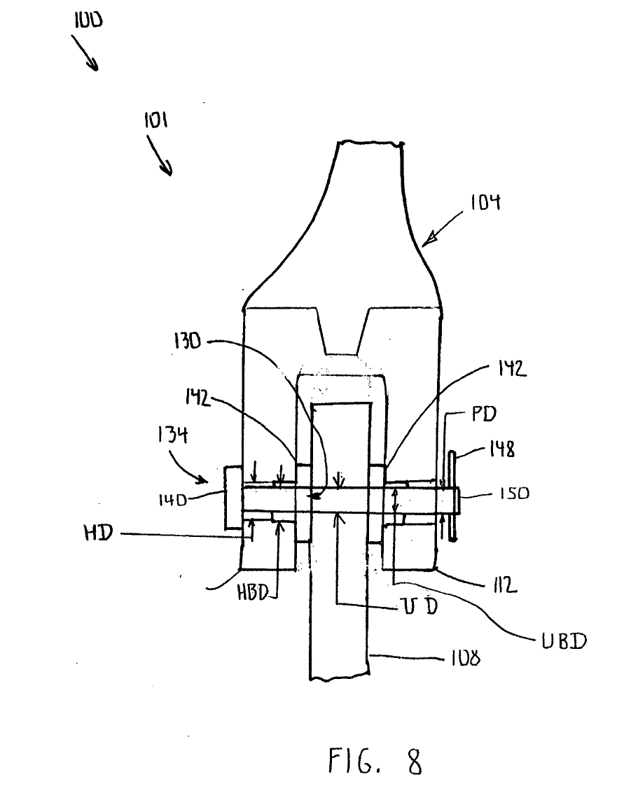

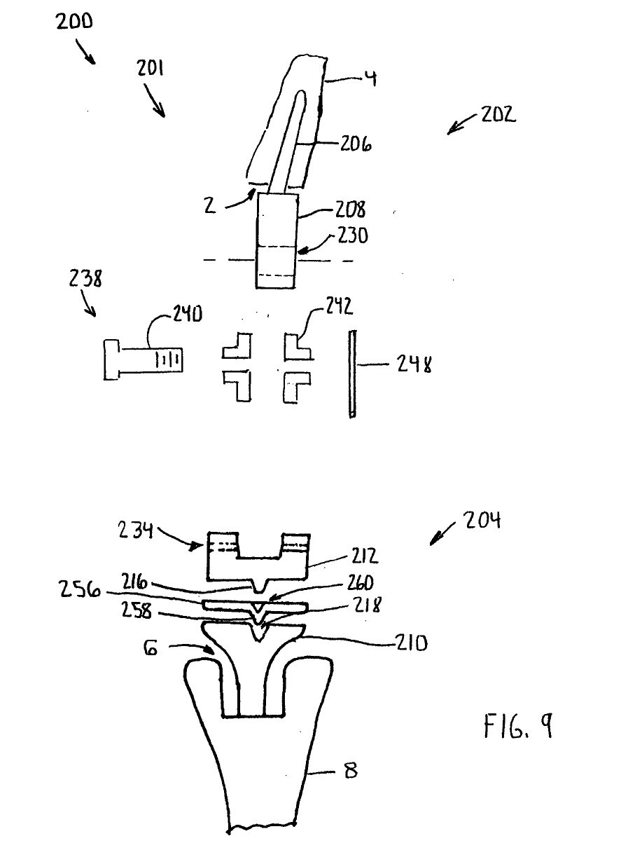

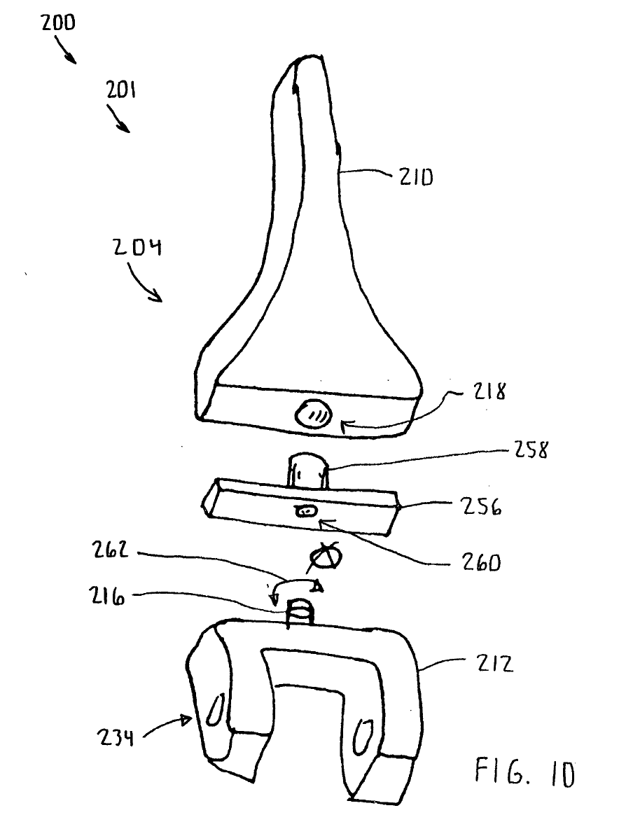

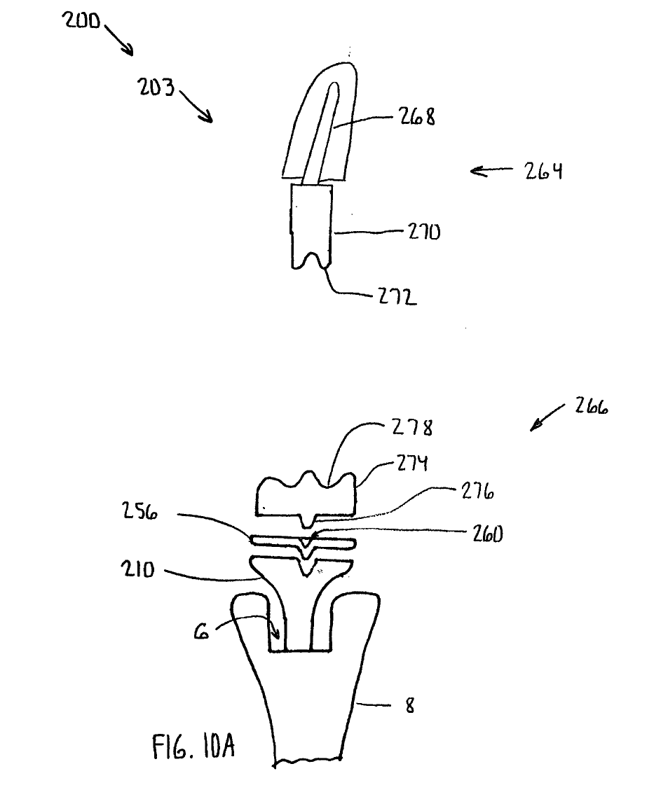

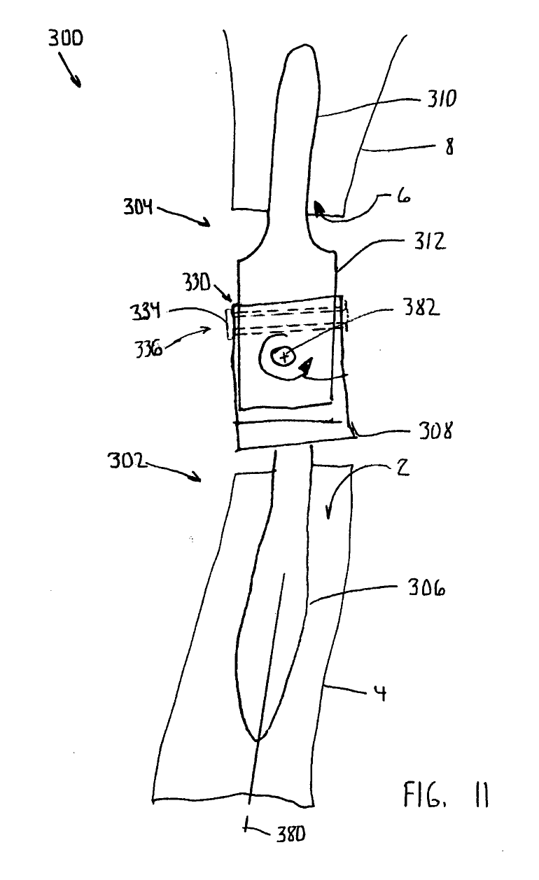

Referring now to FIG. 7, the connector 138 for use with the humeral component 104 and ulnar component 102 to form the elbow prosthesis 100 is shown in greater detail. The connector 138 includes the pin 140 which cooperates with bushings 142 and wire 148 to form the connector 138. The pin 140 includes a body 150 from which head 152 extends. The body 150 defines a cross hole 154 for receiving the wire 148. The body 150 of the pin 140 opening defines pin diameter PD which matingly fits with ulnar diameter UD and with bushing opening UBD. The bushings 142 defined bushing hub diameter HBD which matingly fits with humeral opening diameter HD shown in FIG. 8. Cross-hole 154 of the pin 140 defines a diameter CHD which mates with diameter WD of the wire 148.

Referring now to FIG. 8, the connector 138 is shown in position with the ulnar component 102 and the humeral component 104. The bushings 142 are positioned in humeral opening 134 with each of the bushings 142 having the diameter HBD fitting into diameter HD of the humeral openings 134. The pin 140 is fitted into ulnar opening 130 with the ulnar opening diameter UD being sized for mating fit with body diameter PD of the body 150 of the pin 140. The connector 138 thus matingly interlocks the second portion 112 of the humeral component 104 with the second portion 108 of the ulnar component 102 to form the semi-unconstrained elbow prosthesis 101.

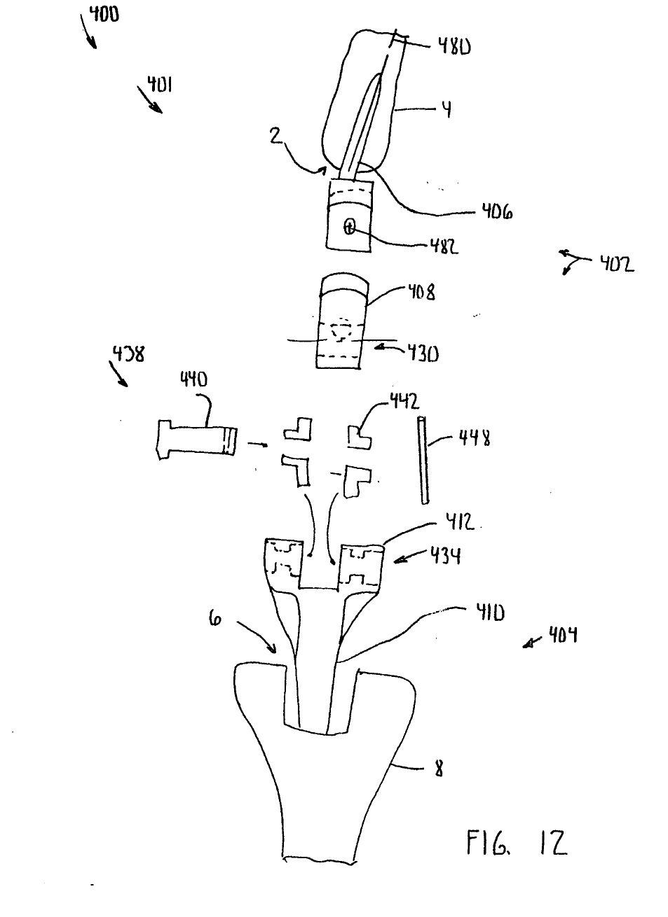

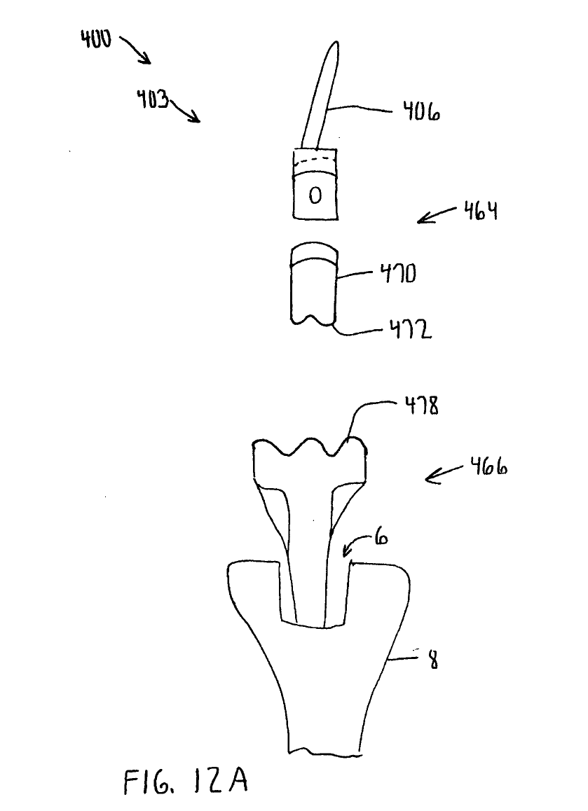

Referring now to FIGS. 9, 10 and 10A, another embodiment of the present invention is shown as elbow prosthesis 200. Elbow prosthesis 200 includes a semi-constrained elbow prosthesis 201 as is shown in FIGS. 9 and 10 and an unconstrained elbow prosthesis 203 as is shown in FIG. 10A.

Referring now to FIG. 9, the semi-constrained elbow prosthesis 201 includes an ulnar component 202, a humeral component 204, and a connector 238 for operatively connecting the ulnar component 202 to the humeral component 204.

The ulnar component 202 includes a stem portion 206 which is fitted into cavity 2 of the ulna 4. An articulating portion 208 extends from the stem portion 206 to form the ulna component 202. The articulating portion 208 defines a transverse opening 230.

The humeral component 204 includes a stem portion 210 which is fitted into cavity 6 of the humerus 8. The humeral component 204 is different than the humeral component 104 of the prosthesis 100 of FIGS. 1-7 in that the humeral component 204 further includes a bearing 256. The bearing 256 serves to assist in providing for a mobile bearing or a rotatable humeral component 204. The bearing 256 includes a protrusion 258 which is fitted into cavity 218 formed in the stem portion 210.

The bearing 256 is positioned between the stem portion 210 and articulating portion 212 of the humeral component 204. The bearing 256 further defines a bearing cavity 260 which mates with protrusion 216 extending from articulating portion 212 of the humeral component 204. The articulating portion 212 defines transverse opening 234 for receiving the connector 238.

The connector 238 includes a pin 240 which cooperates with bushings 242 and wire 248 to form the connector 238. The pin 240 fits through opening 230 of the ulnar component 202 and through the opening 234 of the articulating portion 212 of the humeral component 204 to form the semi-constrained elbow prosthesis 201.

Referring now to FIG. 10, the humeral component 204 of the semi-constrained elbow prosthesis 201 is shown in greater detail. The humeral component 204 includes the stem component 210, the bearing 256, and the articulating component 212. In order to provide the rotation in the direction of arrow 262 between the articulating component 212 and the stem component 210, the bearing 256 is permitted to rotate with at least one of the stem component 210 and the articulating component 212. For example, the cavity 218 formed in the stem component 210 may be rotatably fitted with the bearing protrusion 258. Alternatively, the bearing protrusion 258 and the cavity 218 may define a rigid connection.

Alternatively or in addition, the bearing cavity 260 of the bearing 256 may be rotatably fitted to the protrusion 216 of the articulating component 212 to permit the bearing 256 to rotate with respect to the stem component 210. It should be appreciated that alternatively the protrusion 216 and the bearing cavity 260 may represent a rigid connection.

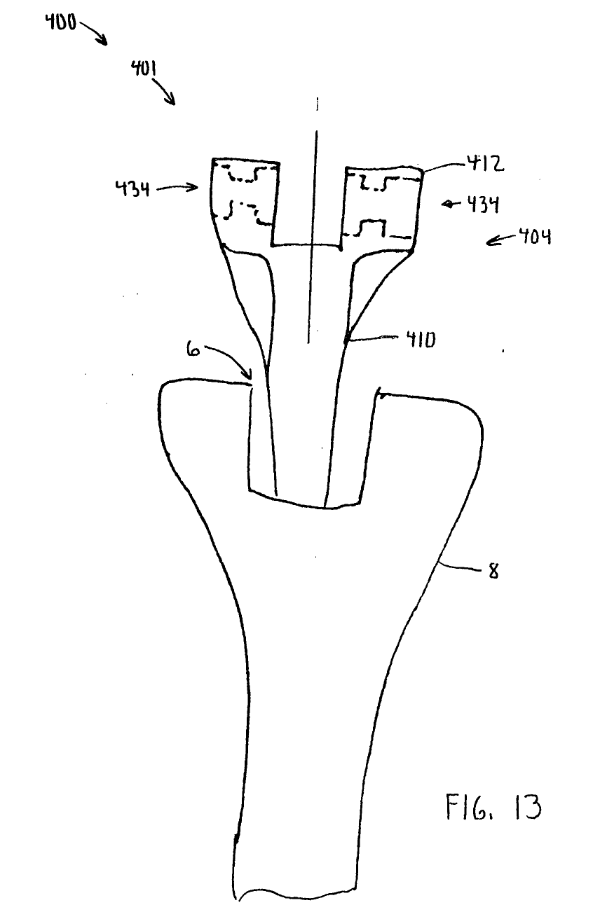

Referring now to FIG. 10A, an unconstrained prosthesis 203 of the prosthesis 200 is shown. The unconstrained prosthesis 203 includes an ulnar component 264 which is in unconstrained cooperation with humeral component 266. The ulnar component 264 is similar to the ulnar component 202 of FIGS. 10 and 11 except that the ulnar component 264 includes an articulating portion 270 which is different than the articulation portion 208 of the ulnar component 202.

The ulnar component 264 includes a stem component 268 which is similar to the stem portion 206 of the ulnar component 202 of FIGS. 10 and 11. The articulation portion 270 of the ulnar component 264 includes an articulating surface 272 which mates with articulating surface 278 of articulation portion 274 of the humeral component 266. The humeral component 266 includes stem portion 210 which is connected to bearing 256.

The articulating portion 274 of the humeral component 266 includes a protrusion 276 which cooperates with bearing cavity 260 of the bearing 256. The articulating surface 272 of the ulnar component 264 cooperates with the articulating surface 278 of the humeral component 266 to provide the unconstrained prosthesis 203.

Referring now to FIG. 11, yet another embodiment of the present invention, is shown as elbow prosthesis 300. The elbow prosthesis 300 includes an ulnar component 302. The ulnar component 302 includes a first portion 306 of the ulnar component 302 which is implantable in cavity 2 formed in the ulna 4. The ulnar component 302 further includes a second portion 308 of the ulnar component 302. The first portion 306 of the ulnar component 302 defines a longitudinal axis 380 of the first portion 306. The second portion 308 of the ulnar component 302 is designed to be rotatable connected to the first portion 306 of the ulnar component 302 about an axis 382 which is normal or perpendicular to the longitudinal axis 380.

The elbow prosthesis 300 further includes a humeral component 304. The humeral component 304 includes a first portion 310 of the humeral component 304. The first portion 310 is implantable in a cavity 6 formed in the humerus 8. The humeral component 304 further includes a second portion 312. The second portion 312 of the humeral component 304 is operably connected to the second portion 308 of the ulnar component 302.

The humeral component 304 may be connected to ulnar component 302 in any suitable manner. For example, a connector 334 may be used and fitted to an ulnar opening 130 and to a humeral opening 336 to provide for a pivoting connection of the humeral component 304 to the ulnar component 302.

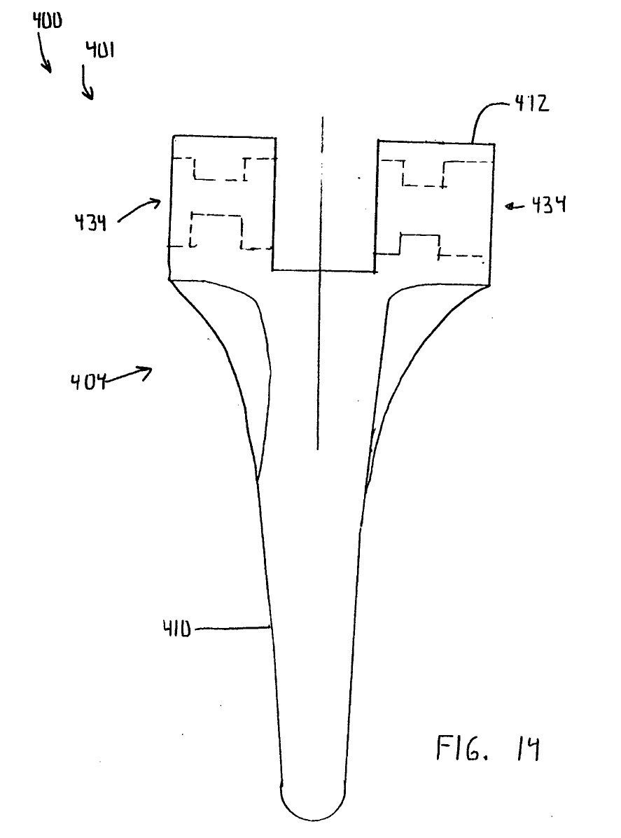

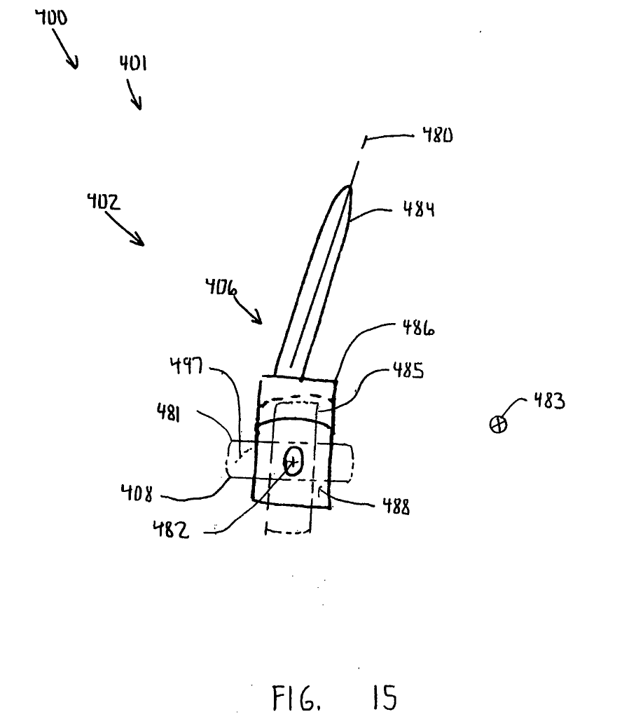

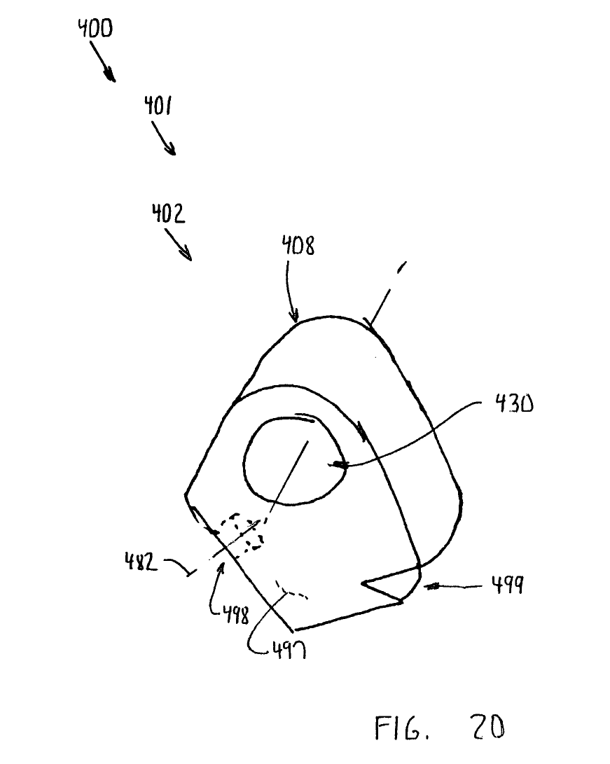

Referring now to FIGS. 12-21, yet another embodiment of the present invention is shown as semi-constrained elbow prosthesis 401 of elbow prosthesis 400. The elbow prosthesis 400 includes an ulnar component 402. The ulnar component 402 includes a first portion or stem 406 which is implantable in a cavity 2 formed in the ulna 4. The ulnar component 402 also includes a second or articulating portion 408. The first portion 406 of the ulnar component 402 defines a longitudinal axis 480 of the ulnar stem portion 406. The ulnar articulating portion 408 of the ulnar component 402 is rotatably connected to the ulnar stem portion 406 of the ulnar component 402 about an axis 482 which is normal to the longitudinal axis 480.

The semi-constrained elbow prosthesis 401 also includes a humeral component 404. The humeral component 404 includes a first portion or stem portion 410 which is implantable in a cavity 6 of the humerus 8. The humeral component 404 also includes a second portion or articulating portion 412. The articulating portion of the humeral component 404 is operably connected to second or articulating portion 408 of the ulnar component 402.

The ulnar component 402 may be operably connected to humeral component 404 in any suitable fashion. For example, and is shown in FIG. 12, the elbow prosthesis 401 is a semi-constrained prosthesis. In the semi-constrained prosthesis 401 the ulnar component 402 is pivotally connected to the humeral component 404.

For example, and is shown in FIG. 12, the semi-constrained elbow prosthesis 401 further includes a connector 438 for pivotally connecting the ulnar component 402 to the humeral component 404. The connector 438, may as shown in FIG. 12, include a pin 440 and a pair of spaced apart bushings 442 which cooperate with a wire 448 as well as with an opening 430 formed in the articulating portion 408 of the ulnar component 402 and with opening 434 formed in the articulating component 412 of the humeral component 404 to provide the pivoting motion of the semi-constrained elbow prosthesis 401.

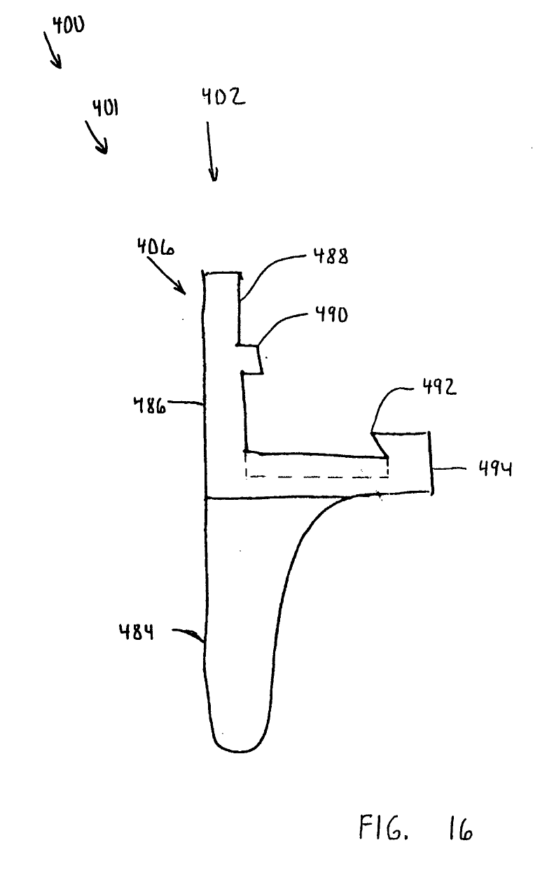

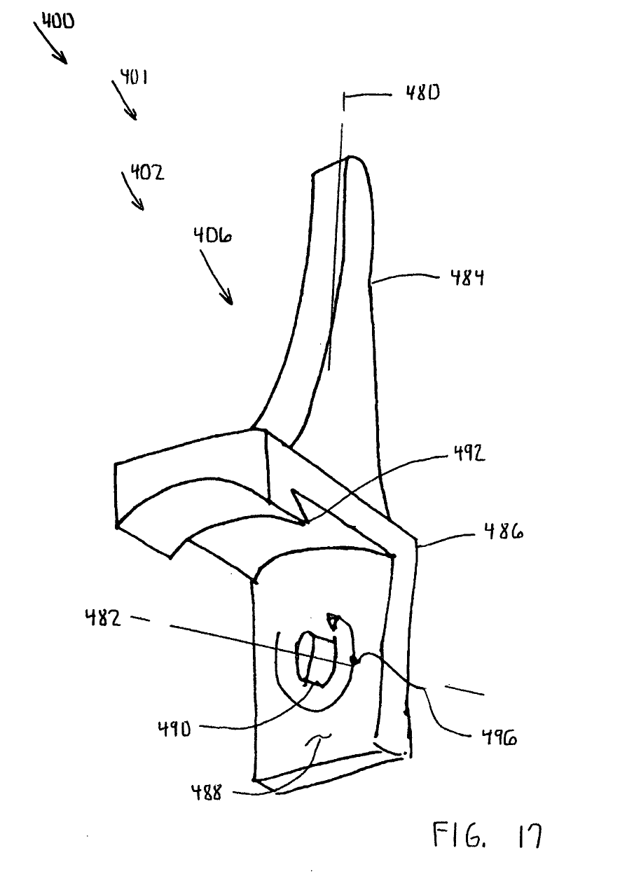

Referring now to FIGS. 13 and 14, the humeral component 404 is shown in greater detail. The humeral component 404 includes the stem portion 410 which is fitted into cavity 6 of the humerus 8 as well as an articulating portion 412 which extends from the stem portion 410 of the humeral component 404. As shown in FIGS. 13 and 14, the stem portion 410 and the articulating portion of the humeral component 404 are integral with each other. It should be appreciated that the stem portion 410 and the articulating portion 412 may be separate components.

The articulating portion 412 of the humeral component 404 defines a pair of spaced apart transverse openings 434 for receiving the connector 438 of FIG. 12.

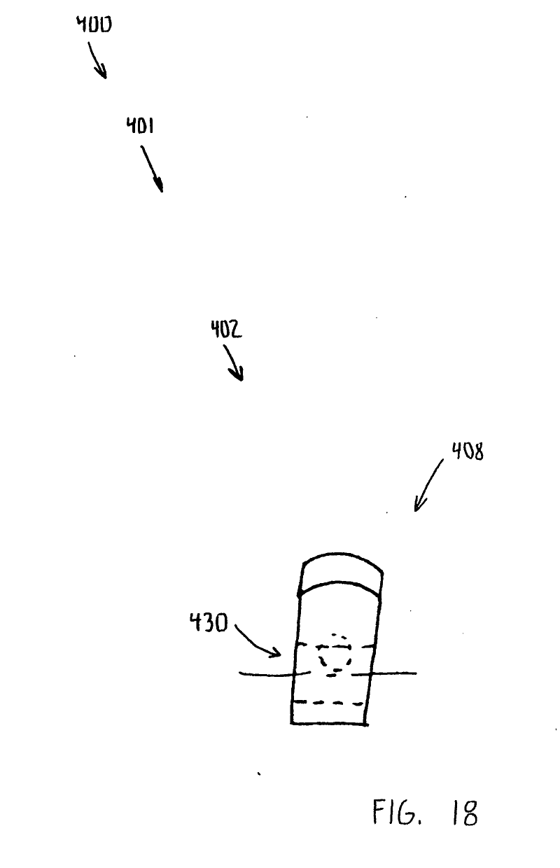

Referring now to FIGS. 15, 16, and 17 the ulnar stem portion 406 of the ulnar component 402 is shown in greater detail. The ulnar stem component 406 includes a stem 484 which defines the longitudinal axis 480 of the ulnar stem component 406. Extending from the stem 484 is the ulnar stem component body 486. The ulnar stem component body 486 defines a support face 488 from which plug 490 extends. The body 486 further defines a lip 492 which extends from transverse member 494 of the body 486. The plug 490 and the lip 492 serve to contain the ulnar-articulating component 408 to rotate about transverse axis 482 of the ulnar stem portion 406 in the direction of arrows 496.

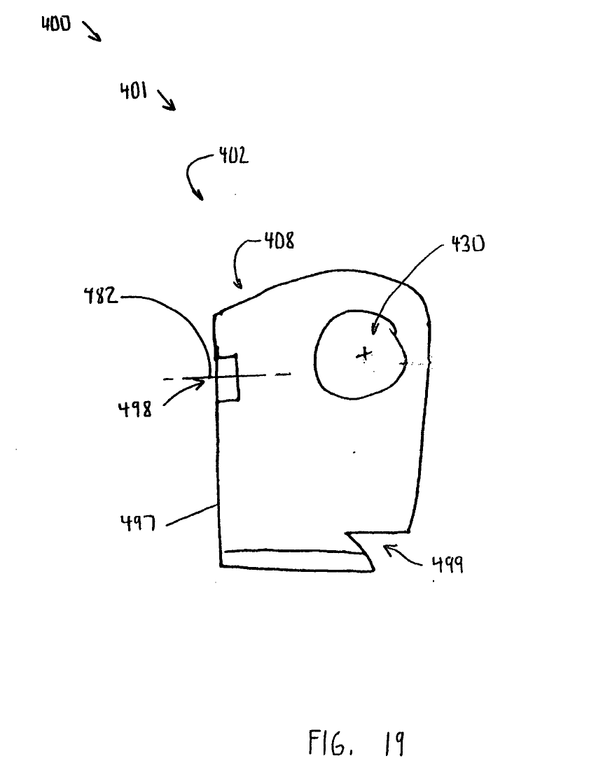

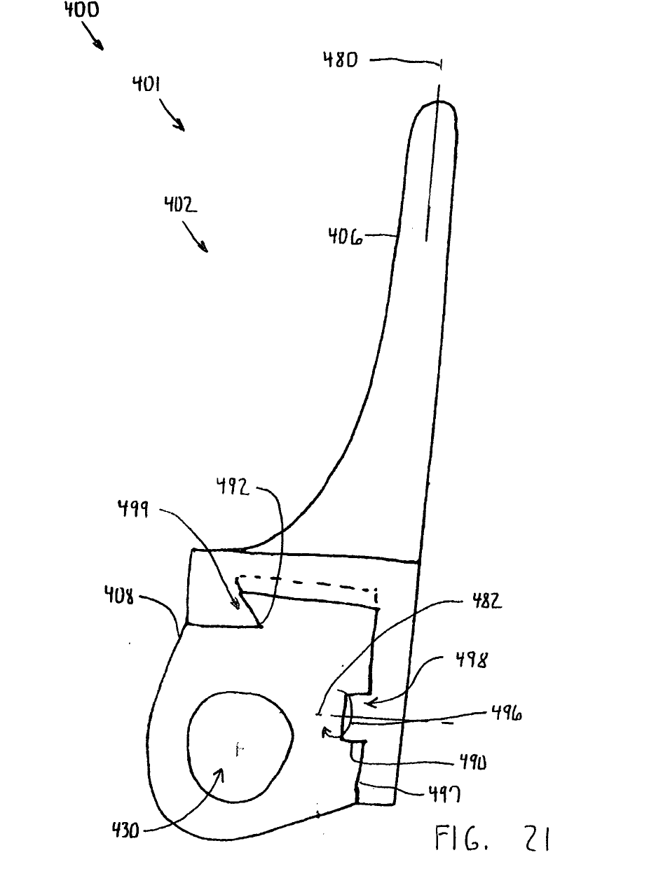

Referring now to FIGS. 18, 19 and 20, the ulnar articulating portion 408 of the ulnar component 402 of the semi-constrained prosthesis 401 is shown in greater detail. The ulnar articulating portion 408 includes a support surface 497 for cooperation with support surface 488 of the ulnar stem portion 406. A pivot hole 498 is formed in articulation portion support surface 497. The pivot hole 498 is adapted to receive the plug 490 of FIG. 16 for rotation therewith. The ulnar articulating portion 408 further defines a groove 499 adapted to mate with the lip 492 of the ulna stem portion 406.

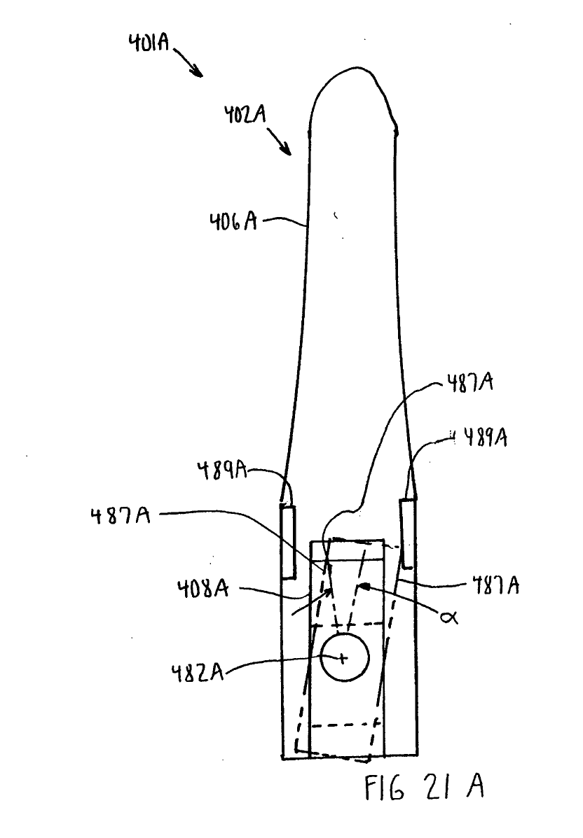

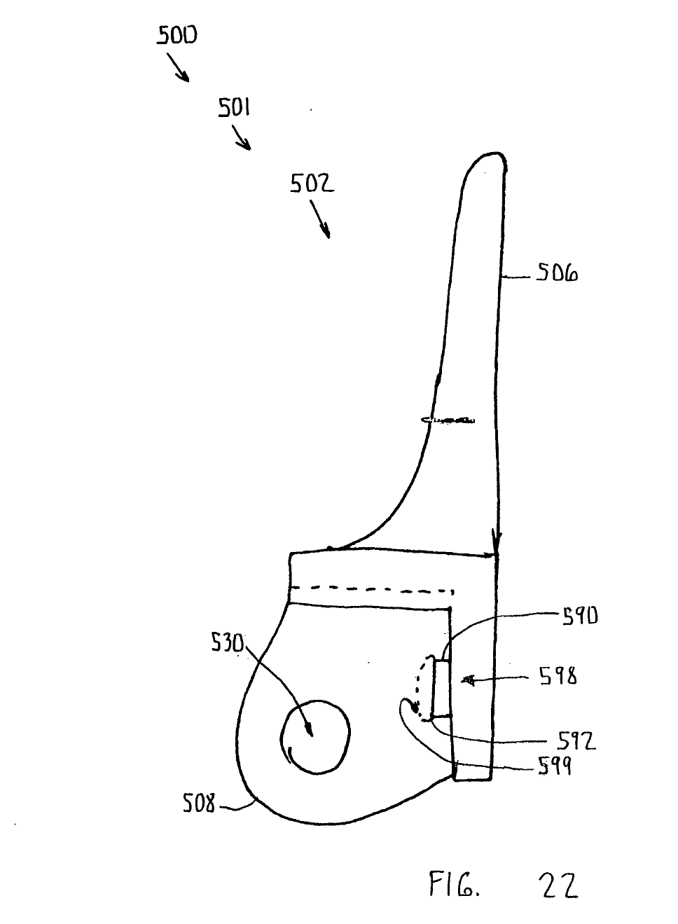

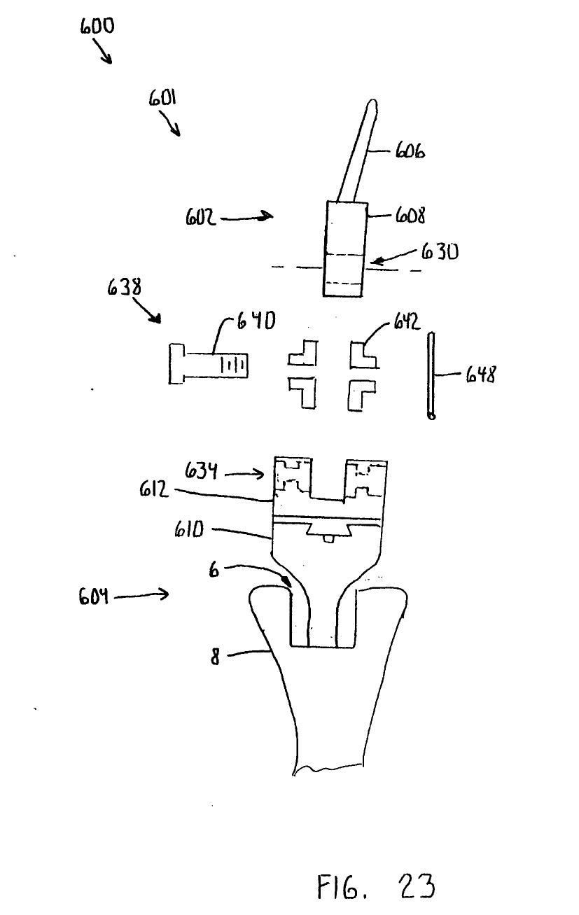

Referring now to FIG. 21, the semi-constrained ulnar component 402 is shown in greater detail. The ulnar component 402 of semi-constrained elbow prosthesis 401 includes the stem portion 406 to which the articulating portion 408 is pivotally connected.

Pivot hole 498 in the support surface 497 of the ulnar articulating component 408 receives plug 490 of the stem portion 406 constraining the articulating portion 408 in a pivoting position about axis 482. Lip 492 of the stem portion 406 engages with groove 499 formed in the articulating portion 408 to constrain articulating portion 408 with respect to the stem portion 406.

Referring again to FIG. 15, the articulating portion 408 is shown in phantom being assembled unto the ulnar stem component 406 to form the ulnar component 402. The ulnar articulating portion 408 is shown in phantom in first position 481 in a position generally normal or perpendicular to the longitudinal axis 480 of the ulnar stem component 406. From the first position 481, the ulnar articulating portion 408 may be advanced in the direction of arrow 483 until the support surface 497 of the ulnar articulating portion 408 rests against support surface 488 of the ulnar stem portion 406. The ulnar-articulating portion 408 is then rotated from first position 481 to second position 485 to provide for the ulnar component 402.

Referring now to FIG. 12A, unconstrained elbow prosthesis 403 of the elbow prosthesis 400 is shown. The unconstrained elbow prosthesis 403 includes an ulnar unconstrained assembly 464 which mates in an unconstrained fashion with humeral component 466. The humeral component 466 is similar to the humeral component 406 of FIGS. 12-21 except that the humeral component 466 includes an articulating surface 478 for unconstrained cooperation with articulating surface 472 of the ulnar unconstrained assembly 464.

The ulnar-unconstrained assembly 464 includes the ulnar stem portion 406 which mates with ulnar unconstrained portion 470. The ulnar unconstrained portion 470 is similar to the ulnar semi-constrained articulating portion 408 of the ulnar component 402 of the FIGS. 12-21 except the ulnar unconstrained articulating portion 470 includes an articulating surface 472 for cooperation with the articulating surface 478 of the humeral component 466.

Referring now to FIG. 21A, yet another embodiment of the present invention is shown as semi-constrained elbow prosthesis 401A. The elbow prosthesis 401A is similar to the elbow prosthesis 401 of FIGS. 12-21 except that the ulnar prosthesis 401A includes ulnar component 402A which is somewhat different than the ulnar component 402 of FIGS. 12-21.

For example, and is shown in FIG. 21A, the ulnar component 402A includes a stem portion 406A and an articulating portion 408A. Stem portion 406A of the ulnar component 402A includes stops 489A which are positioned on the stem portion 406A to limit the rotation of the articulating portion 408A about the first axis 482A.

In fact, and is shown in FIG. 21A, the stops 489A cooperate with faces 487A of the ulnar articulating component 408A to limit the rotation of the articulating portion 408A with respect to the stem portion 406A for example, to a pivoting angle α. α may, for example, be from 10 to 60°.

According to the present invention and referring now to FIG. 22, yet another embodiment of the present invention is shown as elbow prosthesis 500. The elbow prosthesis 500 is shown in FIG. 22 in the form of a semi-constrained elbow prosthesis 501. The semi-constrained elbow prosthesis 501 includes an ulnar component 502 which mates with a humeral component (not shown) similar to humeral component 404 of FIGS. 12-21. The semi-constrained elbow prosthesis 501 further includes a connector (not shown) similar to the connector 438 of the elbow prosthesis 400 of FIGS. 12-21.

The ulnar component 502 includes a stem portion 506 as well as an articulating portion 508. The stem portion 506 is similar to the stem portion 406 of the elbow prosthesis 400 of FIGS. 12-21 but includes a plug 590 which is somewhat different than the plug 490 of the stem portion 406 of the elbow prosthesis 400 of FIGS. 12-21. The plug 590 further includes a rim 592 extending from the plug 590. The stem portion 506 unlike the stem portion 406 of the elbow prosthesis 400 of FIGS. 12-21 does not include a lip.

The articulating portion 508 of the ulnar component 502 of the semi-constrained elbow prosthesis 501 of FIG. 22, is somewhat similar to the articulating portion 408 of the elbow prosthesis 400 of FIGS. 12-21 except that the articulating portion 508 includes a pivot hole 598 which further defines a recess 599 for cooperation with the rim 592 of the plug 590 of the stem portion 506 of the ulnar component 502. The articulating portion 508 defines an ulnar opening 530 for receiving the connector (not shown) which cooperates with the ulnar component (not shown) to form the semi-constrained elbow prosthesis 501.

Referring now to FIGS. 23-30, yet another embodiment of the present invention is shown as elbow prosthesis 600. Elbow prosthesis 600 is similar to the elbow prosthesis 100 of FIGS. 1-8 except that the elbow prosthesis 600 uses a different rotation mechanism for the humeral component in that the elbow prosthesis 600 uses a lip/groove or dovetail connection.

Referring now to FIG. 23, the semi-constrained elbow prosthesis 601 of the elbow prosthesis 600 is shown. The semi-constrained elbow prosthesis 601 includes an ulnar component 602 which is operably connected to a humeral component 604. The ulnar component 602 includes a stem portion 606 from which an articulating portion 608 extends. The articulating portion 608 defines a transverse opening 630 therein.

The humeral component 604 includes a humeral stem portion 610 from which a humeral semi-constrained articulating portion 612 extends. The humeral semi-constrained articulating portion 612 defines a transverse opening 634 therethrough.

A connector 638 as shown in FIG. 23 includes a pin 640 which is slidably receivable in a pair of spaced apart bushings 642. The connector 638 further includes a wire 648 which is secured to the pin 640.

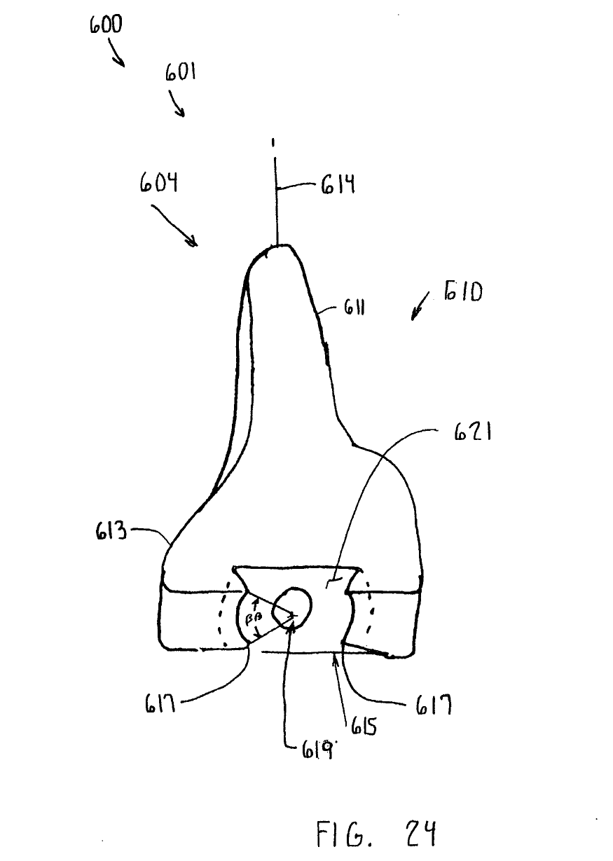

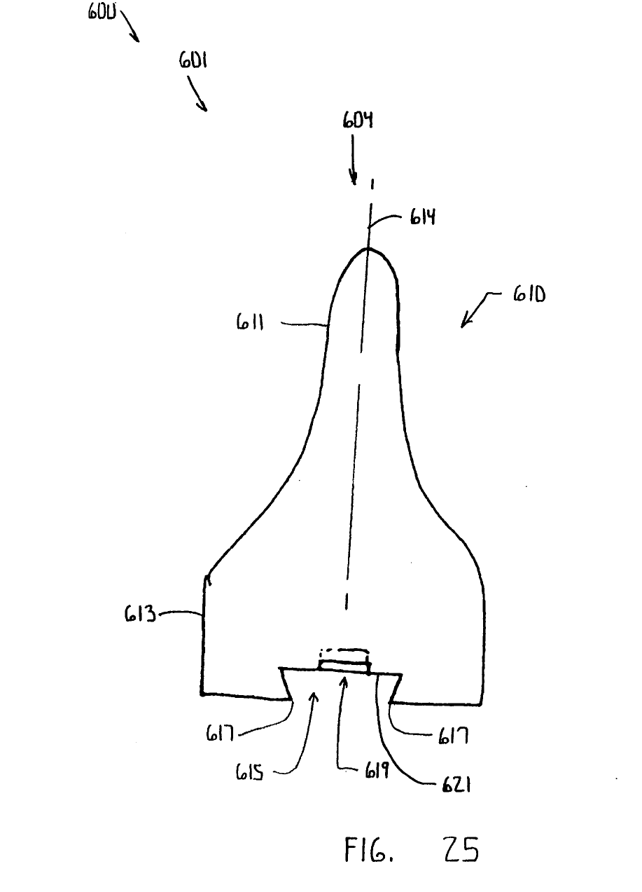

Referring now to FIGS. 24 and 25, the stem portion 610 of the humeral component 604 of the semi-constrained elbow prosthesis 601 is shown in greater detail. The humeral stem 610 includes a distal stem 611 from which a body 613 extends. The body 613 defines a cavity 615 opposed to the stem 611. The cavity 615 is formed from lips 617 formed in the body 613. A central hole 619 is formed in the cavity 615. The stem 611 defines a longitudinal axis 614 which, as shown in FIGS. 24 and 25, defines the center of the central hole 619 and the center of the opposed lips 617. The body 613 of the humeral stem portion 610 defines a support surface 621.

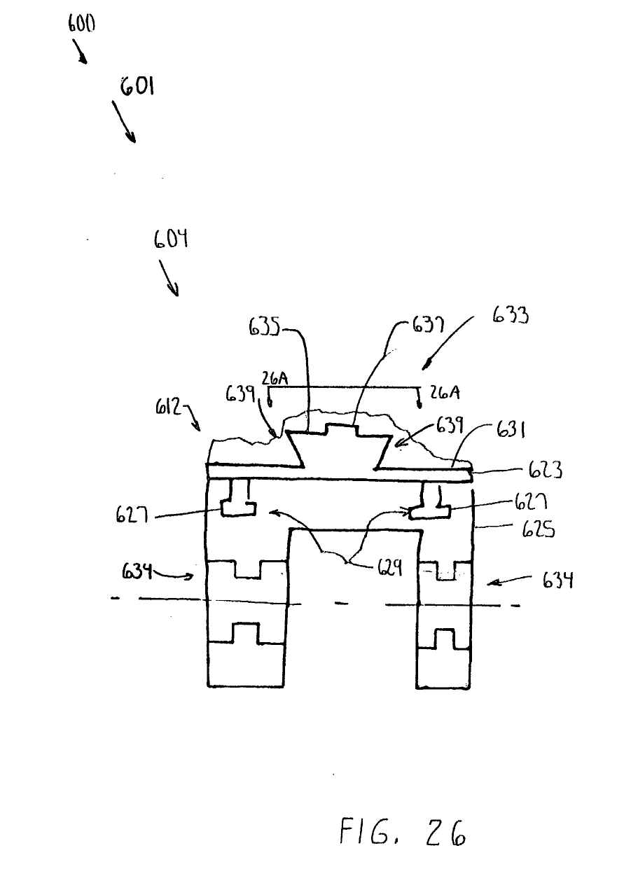

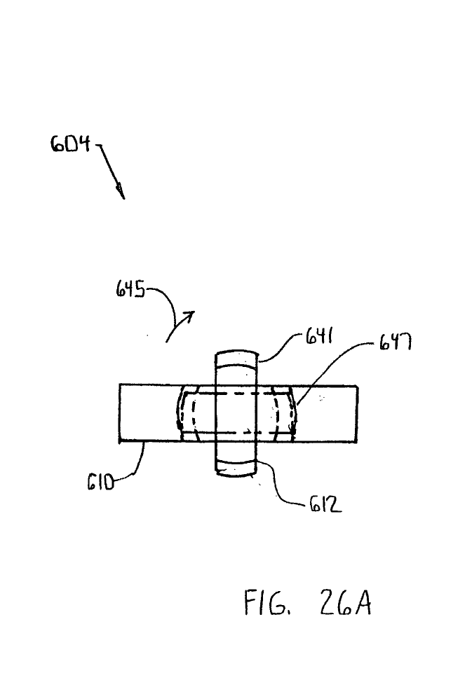

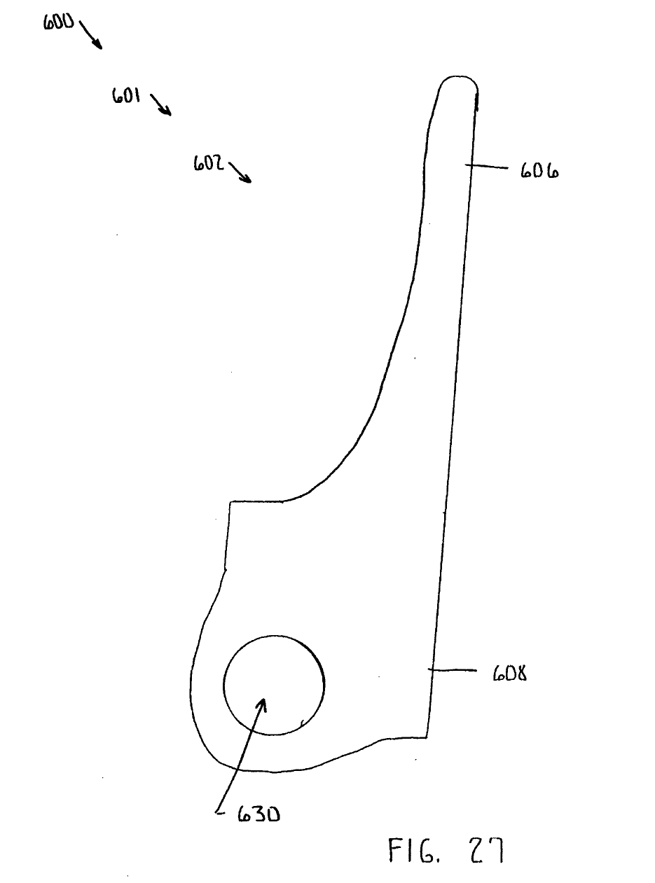

Referring now to FIG. 26, articulating portion 612 of the humeral component 604 of the semi-constrained elbow prosthesis 601 is shown in greater detail. The humeral articulating portion 612 may be integral or may as shown in FIG. 26 be made of more than one component. For example, the humeral articulating portion 612 may include a base 623 and a body 625 which extends from the base 623.

The base 623 and the body 625 may be made of any suitable, durable material. For example, the base 623 and body 625 may be made of a metal or plastic. The base 623 may be made of a plastic and the body 625 may be made of metal. The base 623, if made of a plastic, may provide for the mobile bearing or rotation capability.

The base 623 may include a pair of spaced apart pegs 627 which mate with cavities 629 formed in the body 625. The base 623 of the humeral articulating portion 612 may define an end face 631 from which dovetail 633 centrally extends. The dovetail 633 defines a support surface 635 as well as a central pivot 637. The body 625 may define a pair of spaced apart transverses openings 634 for cooperation with the bushings 642 of FIG. 23. The dovetail 633 defines opposed recesses 639 for cooperation with the lips or protrusions 617 formed in the humeral stem portion 610 of FIG. 24.

Referring now to FIG. 26A, the humeral articulating portion 612 may be assembled unto the humeral stem portion 610 of the humeral component 604 in any suitable manner. For example, and is shown in FIG. 26A, the humeral articulating portion 612 may be positioned in first position 641 as shown in solid over the humeral stem portion 610. The humeral articulating portion 612 may be advanced in the direction of arrow 645 until the support surface 635 of the dovetail 633 of the articulating portion 612 rests against the support surface 621 of the humeral stem portion 610 (see FIGS. 25 and 26). The pivot 637 of the articulating portion 612 may be fitted into central hole 619 of the humeral stem portion 610. When the surfaces 621 and 635 are in contact, the humeral articulating portion 612 may be rotated in the direction of arrow 645 from first position 641 to second position 647, as shown in phantom, thereby securing the humeral articulating portion 612 to the humeral stem portion 610 to form the humeral component 604.

Referring now to FIG. 27, the humeral component 602 of the semi-constrained elbow prosthesis 601 is shown in greater detail. The ulnar component 602 includes a stem portion 606 from which articulating portion 608 extends. The articulating portions 608 define the transverse opening 630 for receiving the connector 638.

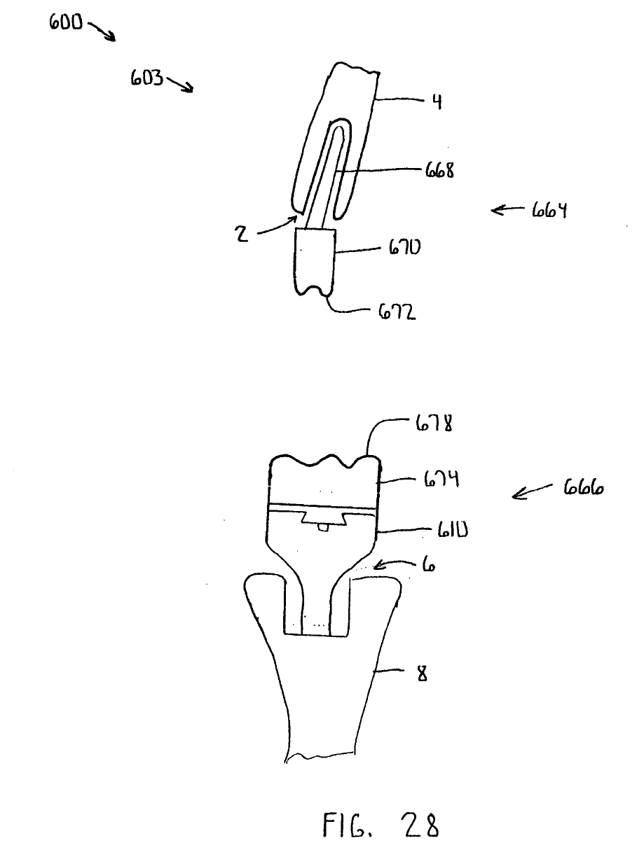

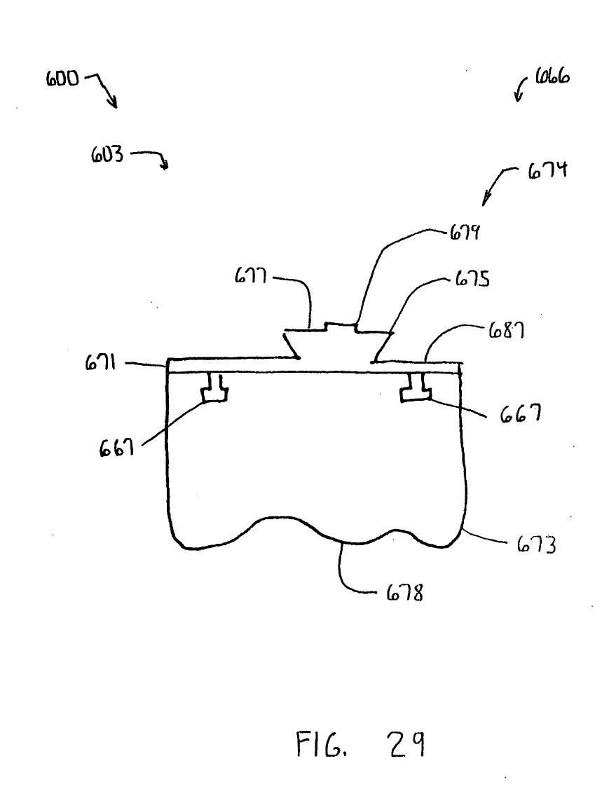

Referring now to FIG. 28, unconstrained elbow prosthesis 603 of the elbow prosthesis 600 is shown. The unconstrained elbow prosthesis 603 includes an ulnar unconstrained component 664 which cooperates in an unconstrained fashion with unconstrained humeral component 666 to form the unconstrained elbow prosthesis 603.

The unconstrained ulnar component 664 includes an ulnar stem portion 668 which is fitted into the cavity 2 of the ulna 4. An ulnar articulating portion 670 extends from the ulnar stem portion 668. The ulnar articulating portion 670 defines ulnar articulating surface 672.

The humeral unconstrained component 666 includes a humeral stem portion 610 which is fitted into cavity 6 of the humerus 8. Unconstrained articulating portion 674 extends from the humeral stem portion 610. The unconstrained articulating portion 674 defines humerus articulating surface 678 which mates with ulnar articulating surface 672 of the unconstrained ulnar component 664 to form the unconstrained elbow prosthesis 603.

Referring now to FIG. 29, the articulating portion 674 of the humeral component 666 is shown in greater detail. The articulating portion 674 may include a base 671 which is connected to the body 673 to form the unconstrained articulating portion 674. The base 671 and the body 673 may be integral or may, as shown in FIG. 29, be compromised of different components. For example, and as shown in FIG. 29, the base 671 may include a pair of spaced apart pegs 667 for securing the body 671 to the base 673.

The base 671 and the body 673 may be made of any suitable, durable material. For example, the body 673 may be made of a metal and the base 671 may be made of a plastic to provide for a bearing surface for the pivotal connection of the humeral component 666.

The base 671 may include a dovetail 675 centrally positioned and extending from end 687 of the base 671. The dovetail 675 may define a support surface 677 from which a central cylindrical pivot 679 may extend. The dovetail 675 of the humeral unconstrained articulating portion 674 may be similar to the dovetail 633 of the humeral semi-constrained articulating portion 612 so that the humeral articulating portions 612 and 674 may be interchanged.

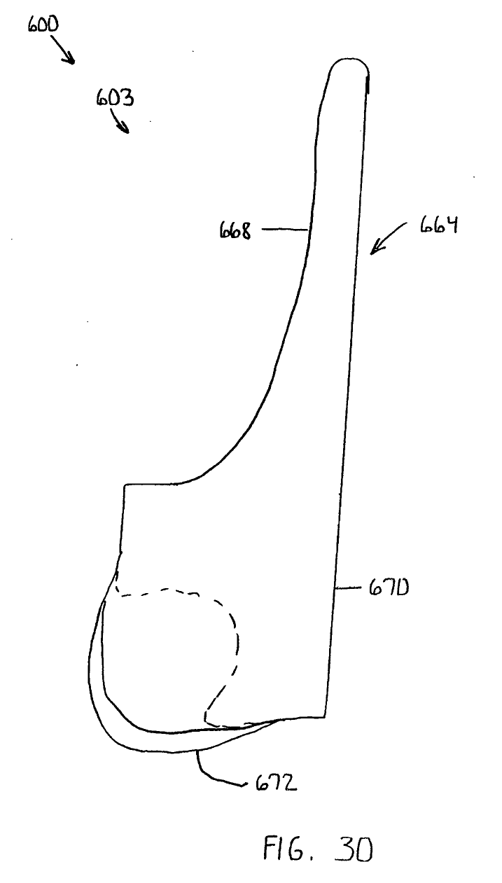

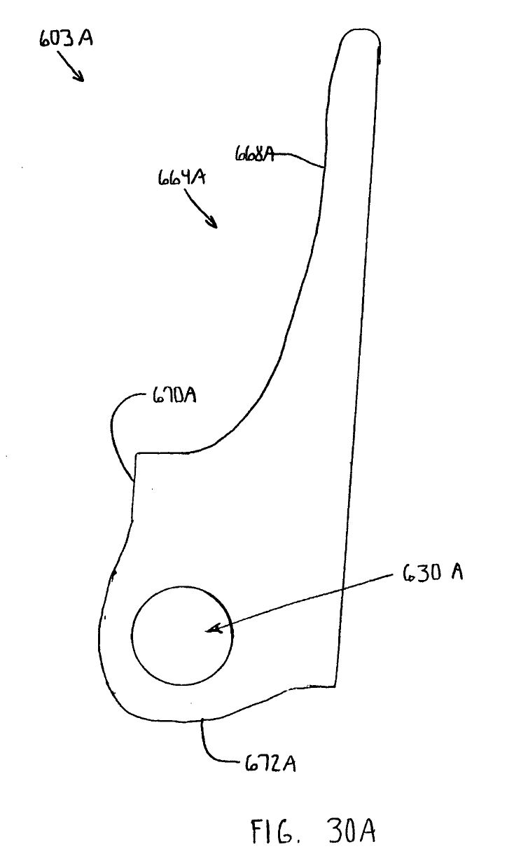

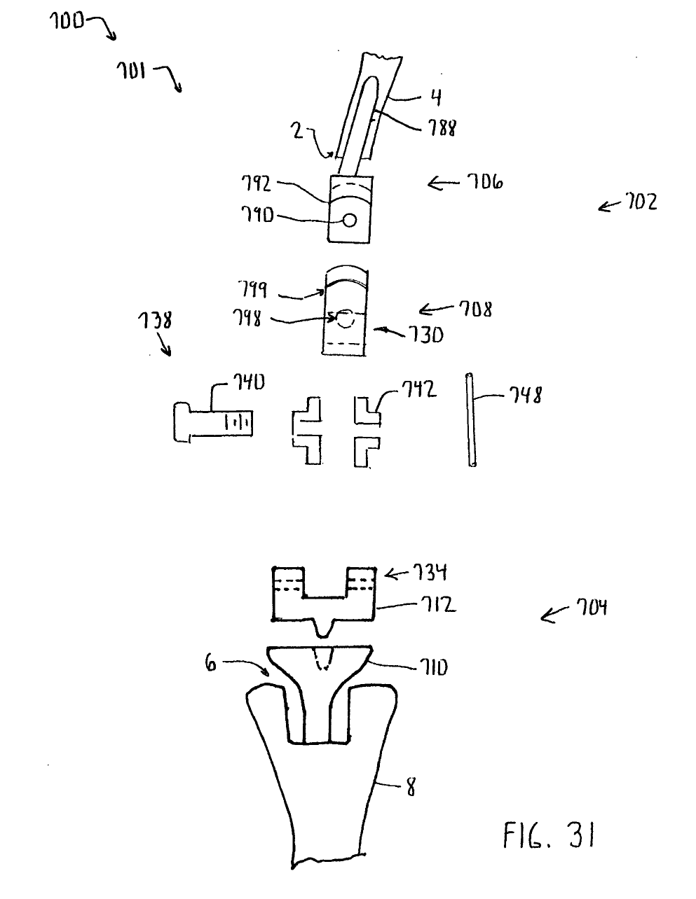

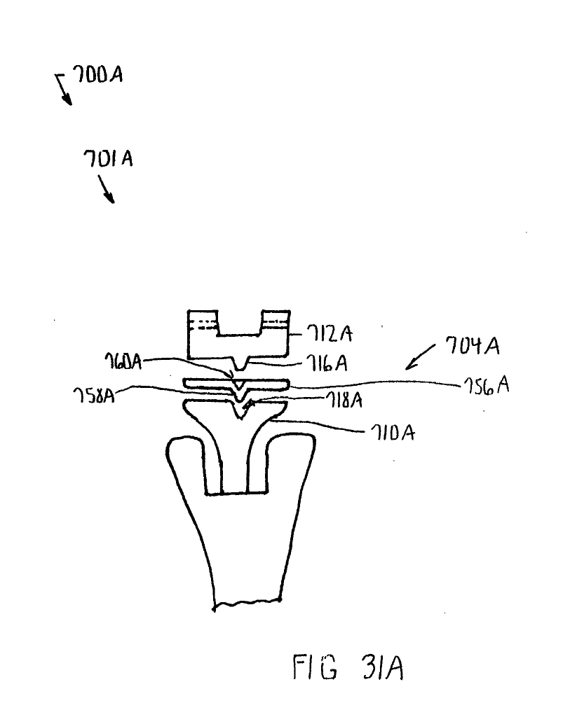

Referring now to FIG. 30, unconstrained ulnar component 664 of the unconstrained elbow prosthesis 603 is shown in greater detail. The unconstrained ulnar component 664 includes a stem portion 668 from which an articulating portion 670 extends. The articulating portion 670 includes articulating surface 672 for cooperation with the articulating surface 678 of the humeral unconstrained component 666.

Referring now to FIG. 30A, yet another embodiment of the present invention is shown as unconstrained elbow prosthesis 603A. The unconstrained elbow prosthesis 603A is similar to the elbow prosthesis 603 of FIGS. 28-30 except that the unconstrained elbow prosthesis 603A includes an ulnar component 664A which is different than the unconstrained ulnar component 664 of the unconstrained elbow prosthesis 603 of FIGS. 28-30. For example, and is shown in FIG. 30A, the ulnar component 664A may be used for both an unconstrained and semi-constrained prosthesis.

As shown in FIG. 30A, the ulnar component 664A includes a stem portion 668A from which an articulating portion 670A extends. The articulating portion 670A includes both an ulnar articulating surface 672A for use in an unconstrained prosthesis as well as a transverse opening 630A for use in a semi-constrained elbow prosthesis.

Referring now to FIG. 31, yet another embodiment of the present invention is shown as elbow prosthesis 700. The elbow prosthesis 700 of FIG. 31 may be used for both rotation about peg shift version as well as for rotation about peg shift carrying angle. The elbow prosthesis 700 of FIG. 31 thus provides both for a mobile bearing configuration on the humeral side of the elbow prosthesis as well as for a mobile bearing prosthesis on the ulnar side of the elbow prosthesis.

Continuing to refer to FIG. 31 semi-constrained elbow prosthesis 701 of the elbow prosthesis 700 is shown. It should be appreciated that the elbow prosthesis 700 of FIG. 31 may be used in an unconstrained version similar to the versions of other embodiments of the present invention in addition to the semi-constrained prosthesis 701 in FIG. 31.

The semi-constrained elbow prosthesis 701 includes an ulnar component 702 which is connected to a humeral component 704 by a connector 738.

The ulnar component 702 includes an ulnar stem portion 706 which includes a part 788 which is fitted into cavity 2 of ulna 4. The ulnar component 702 further includes an ulnar articulating portion 708 which is operably connected to the ulnar stem component 706. The ulnar articulating component 708 is connected to the ulnar stem component by means of a pivot hole 798 extending from the ulnar articulating portion 708, which cooperates with plug 790 formed in the ulnar stem portion 706. A lip 792 formed on the ulna stem portion cooperates with a groove 799 formed in ulna articulating portion 708. The ulna articulating portion 708 defines opening 730 for receiving connector 738.

The humeral component 704 includes a stem portion 710 which is fitted into cavity 6 of the humerus 8. The humeral component 704 includes an articulating portion 712 which is rotatably connected to the stem portion 710. The articulating portion 712 defines a transverse opening 734 for receiving the connector 738.

The connector 738 may, as shown in FIG. 31 include a pin 740, which cooperates with spaced apart bushings 742 to connect the ulnar component 702 with the humeral component 704. The pin 740 may be secured by, for example, wire 748.

Referring now to FIG. 31A, yet another embodiment of the present invention is shown as semi-constrained elbow prosthesis 701A. The elbow prosthesis 701A includes a humeral component 704A which is somewhat similar to the humeral component 704 of the semi-constrained elbow prosthesis 701 of FIG. 31. The humeral component 704A cooperates with an ulnar component (not shown) similar to ulnar component 702. The humeral component 704A and the ulnar component are connected with, for example, a connector (not shown) similar to the connector 738 of the semi-constrained elbow prosthesis 701 of FIG. 31.

The humeral component 704A includes a stem portion 710A. A bearing 756A is rotatably connected to the stem portion 710A. An articulating portion 712A is operably connected to the bearing 756A. At least one of the stem portion 710A, bearing 756A, and articulating portion 712A are rotatably interconnected with each other. It should be appreciated that at least two of the components may be rigidly secured to each other. The stem portion 710A may include, as is shown, a cavity 718A which cooperates with a protrusion 758A formed in the bearing 756A. The bearing 756A may include a bearing cavity 760A which mates with protrusion 716A formed on the articulating portion 712A.

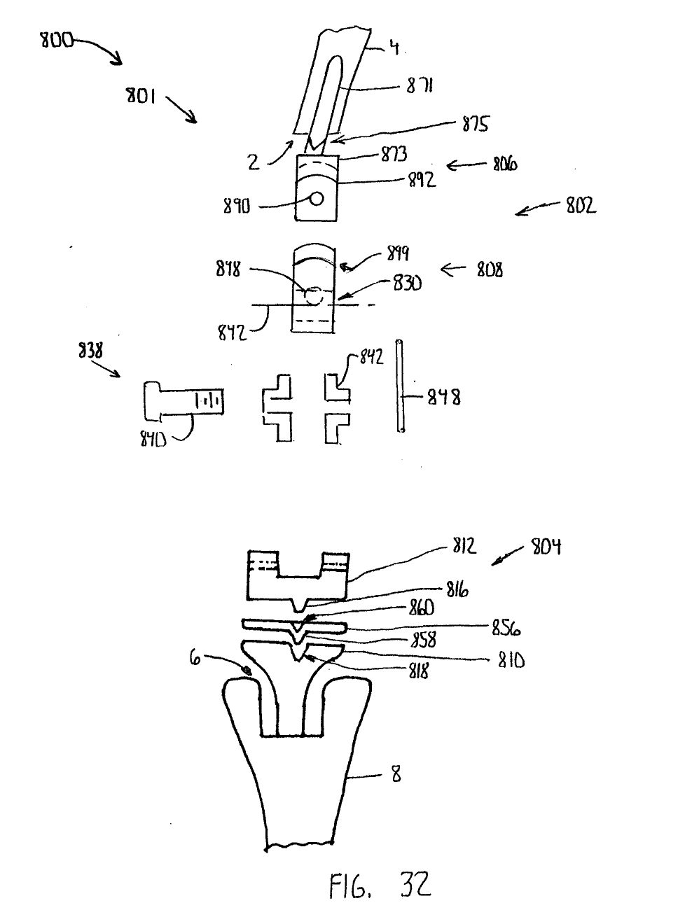

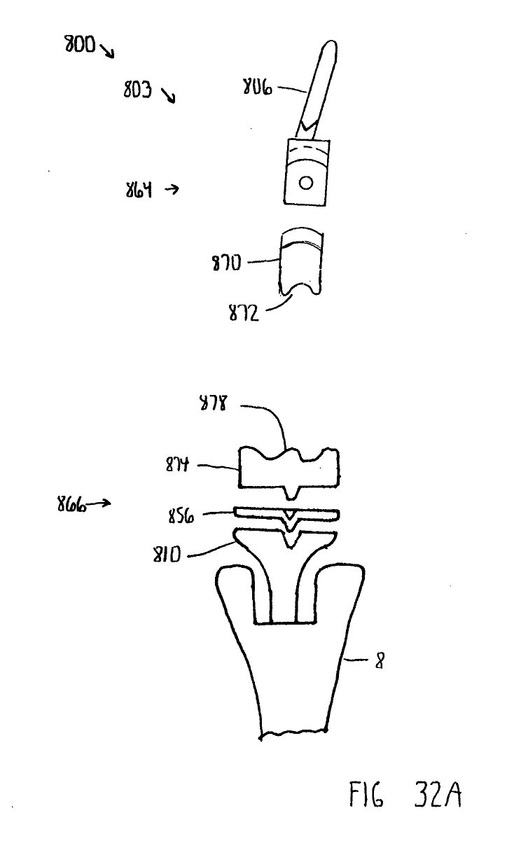

Referring now to FIG. 32, yet another embodiment of the present invention is shown as elbow prosthesis 800. Elbow prosthesis 800 is similar to the prosthesis 700 of FIG. 31 except that the elbow prosthesis 800 includes a modular stem. For example, and referring to FIG. 32, the elbow prosthesis 800 may be used in the form of semi-constrained elbow prosthesis 801. The semi-constrained elbow prosthesis 801 includes an ulnar component 802, which is operably connected to a humeral component 804 by, for example, a connector 838.

The ulnar component 802 includes an ulnar stem portion 806 which is fitted into a cavity 2 of the ulna 4. The ulnar stem portion 806 as is shown in FIG. 32 includes a stem part 871 from which body part 873 extends. The stem part 871 is connected to the body part 873 by means of a connector 875 which may be in the form of a tapered connection. For example, the stem parts 871 may include an external protrusion in a cavity formed in the body part 873.

An ulna bushing portion 808 is pivotally connected to the ulnar stem portion 806. For example, the ulnar-bushing portion 808 includes a pivot hole 898, which cooperates with a plug 890 formed in the ulnar stem portion 806. The ulnar bushing portion 808 may include groove 899 which cooperates with lip 892 formed in the ulnar stem portion 806. The ulnar bushing portion 808 may define a transverse opening 830 which defines rotational axis 842 of the ulnar component 802.

The humeral component 804 includes a stem portion 810 which is fitted into cavity 6 of the humerus 8. A bearing 856 may cooperate with the stem portion 810. An articulating portion 812 is operably connected to the bearing 856. At least one of the stem portion 810, the bearing 856, and the articulating portion 810 is rotatably secured to one of the other components. The bearing 856 may, as is shown in FIG. 32, include a protrusion 858 which mates with cavity 818 formed in the stem portion 810. The bearing 856 may include a bearing cavity 860, which cooperates with protrusion 816 of the articulating portion 812.

The connector 838 may include a pin 840 which cooperates with bushings 842. A wire 848 may be used to secure the pin 840 to the ulnar component 802 and the humeral component 804.

Referring now to FIG. 32A, an unconstrained elbow prosthesis 803 is shown as part of the elbow prosthesis 800. The unconstrained elbow prosthesis 803 includes an unconstrained ulnar component 864, which includes an articulating surface 872, which cooperates with articulating surface 878 of humeral component 866.

The ulnar unconstrained articulating component 864 includes ulnar stem portion 806 to which ulnar articulating portion 870 is rotatably secured. The ulnar articulating portion 870 defines the articulating surface 872.

The humeral component 866 includes the stem portion 810 to which bearing 856 is secured. Articulating portion 874 is secured to the bearing 856 to form the humeral articulating unconstrained component 866. At least two of the stem portion 810, the bearing 856, and the articulating portion 874 are configured for rotation between each other to provide for the mobile bearing feature of the unconstrained elbow prosthesis 803.

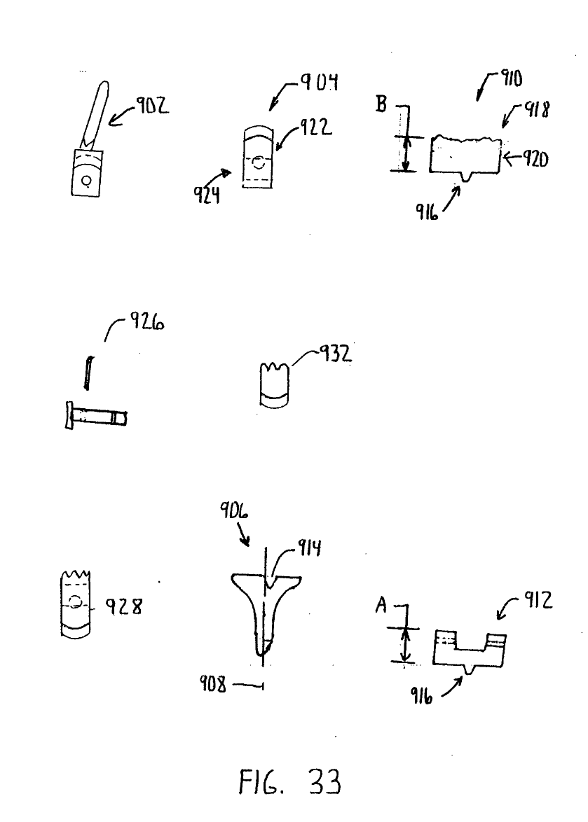

Referring now to FIG. 33, a kit 900 for use in total elbow athroplasty is shown. The kit 900 includes an ulnar stem component 902 for implantation at least partially in the ulna medullary canal. The kit 900 further includes first ulnar component hinge component 904 attachable to the ulnar stem component 902. The kit 900 further includes a humeral stem component 906 for implantation at least partially in the humeral medullary canal. The humeral stem component 906 defines a longitudinal axis 908 of the humeral stem component 906.

The kit 900 also includes a first humeral hinge component 910 which is removably attachable to humeral stem component 906. The first humeral hinge component 910 is also rotatable with respect to the humeral stem component 906 about the longitudinal axis 908.

The kit 900 further includes a second humeral hinge component 912 which is removably attachable to the humeral stem component 906 and rotatable with respect to the humeral stem component 906 about the longitudinal axis 908. The second humeral hinge component 912 has at least one dimension A that is different than dimension B of the first humeral hinge component 910. The kit 900 may further include a second ulnar hinge component 932 attachable to the ulnar stem component 902.

The first humeral hinge component 910 may be, as is shown in FIG. 33, adapted for non-interlockable cooperation with 2^{nd} ulnar hinge component 932.

The second humeral hinge component 912 may, as is shown in FIG. 33, be adapted to be rotatably interlockable with the first ulnar hinge component 904.

The ulnar stem component 902 and the first ulnar hinge component 904 may alternatively be integral with each other. The humeral stem component 906 may define a first tapered connection 914 and the first humeral hinge component 910 and the second humeral hinge component 912 may define a second tapered connector 916. The second connector 916 may be adapted to mate with the first connector 914 of the humeral stem component 906.

The first humeral hinge component 910 may include a hinge portion 918 thereof defining a humeral opening 920 therethrough.

The first ulnar hinge component 904 may further include a hinge portion 922 defining an ulnar opening 924 therethrough. The kit 900 may further include a pin 926 for cooperation with humeral opening 920 and ulnar opening 924.

Kit 900 may further include an ulnar hinge component 928 which is adapted for interlockable cooperation with the second humeral hinge component 912 and for non-interlockable cooperation with the first humeral hinge component 910.

The kit 900 may include the first ulnar hinge component 904 for interlockable cooperation with the humeral hinge stem component 902 as well as including the second ulnar hinge component 932 attachable to the ulnar stem component 902. The second ulnar hinge component 932 may be adapted for non-interlockable hinge cooperation with the first humeral hinge component 910.

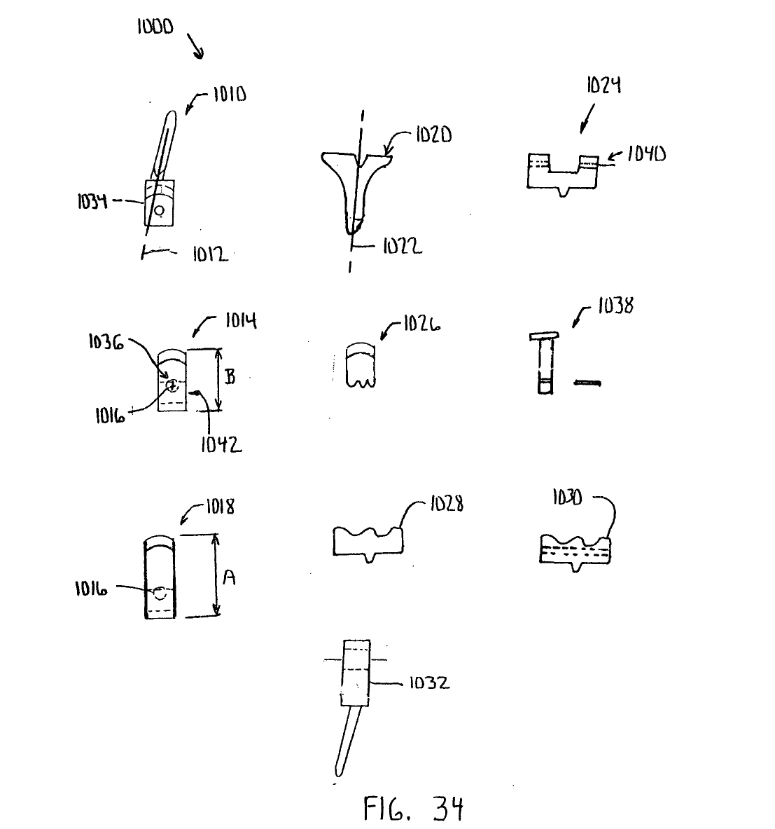

Referring now to FIG. 34, yet another embodiment of the present invention is shown as kit 1000. The kit 1000 is used for use in total elbow athroplasty. The kit 1000 includes an ulnar stem component 1010 for implantation at least partially in the ulnar medullary canal. The ulnar stem component 1010 defines a longitudinal axis 1012 of the ulnar stem 1010.

The kit 1000 further includes a first ulnar hinge component 1014 attachable to the ulnar stem component 1010 for rotatably operation with the ulnar stem component 1010 about an axis 1016 normal to the longitudinal axis 1012 of the ulnar stem component 1010. The kit 1000 further includes a second ulnar hinge component 1018 attachable to the ulnar stem component 1010 for rotatably operation with the ulnar stem component 1010 about an axis 1016 normal to the longitudinal axis 1012 of the ulnar stem component 1010. At least one dimension A of the second ulnar hinge component 1018 is different than dimension B of the first ulnar hinge component 1014.

The kit 1000 further includes a humeral stem component 1020 for implantation or at least partial implantation into the humeral medullary canal. The humeral stem component 1020 defines a longitudinal axis 1022 of the humeral stem component 1020. The kit 1000 further includes a first humeral hinge component 1024 attachable to the humeral stem component 1020 and adapted for cooperation with the first ulnar hinge component 1014 or second ulnar hinge component 1018.

As shown in FIG. 34, the first ulnar hinge component 1014 may be adapted for interlockable cooperation with the first humeral hinge component 1024.

The kit 1000 may further include a third ulnar hinge component 1026 which is adapted for non-interlockable cooperation with a second humeral hinge component 1028. The kit 1000 may further include a unitary humeral hinge component 1030 which is adapted for both unconstrained and semi-constrained articulation with an ulnar component.

The kit 1000 may further include an ulnar component 1032 in which the humeral stem component and the humeral hinge component are integral with each other. The kit 1000 may, for example, be configured such that the ulnar stem component 1010 defines a first connection in the form of, for example, a cylindrical plug 1034. The first ulnar hinge component 1014 and the second ulnar hinge component 1018 may define a second ulnar connector in the form of, for example, pivot hole 1036. The pivot hole 1036 is adapted to mate with the plug 1034.

The kit 1000 may further include a pin 1038 for cooperation with humeral opening 1040 formed in the first humeral articulating component 1024 and the ulnar opening 1042 formed in the first ulnar hinge component 1014.

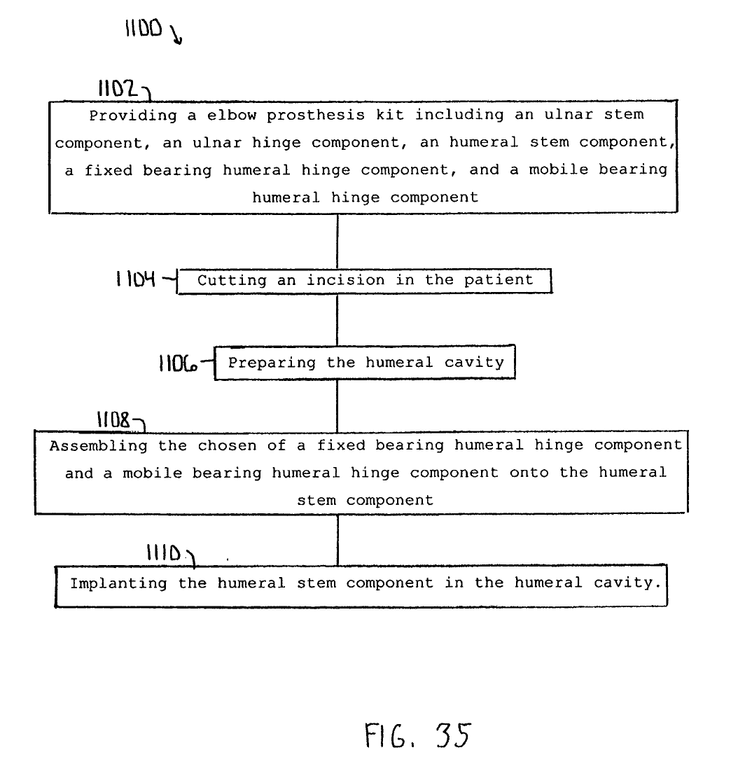

Referring now to FIG. 35, another embodiment of the present invention is shown as surgical procedure method 1100. The method 1100 is used for providing total elbow arthroplasty. The method 1100 includes a first step 1102 in providing an elbow prosthesis kit including an ulnar stem component, an ulnar hinge component, a humeral stem component, a fixed bearing humeral hinge component, and a mobile bearing humeral hinge component. The method 1100 further includes a second step 1104 of cutting an incision in the patient and a third step 1106 of preparing the humeral cavity.

The method 1100 further includes a fourth step 1108 of assembling one of the fixed bearing humeral hinge components and a mobile bearing humeral hinge component onto the humeral stem component. The method 1100 further includes a fifth step 1110 of implanting a humeral stem component into the humeral cavity.

Although the present invention and its advantages have been described in detail, it should be understood that various changes, substitutions, and alterations can be made therein without departing from the spirit and scope of the present invention as defined by the appended claims.

We claim:

1. An elbow prosthesis comprising:
an ulnar component, said ulnar component including a first portion thereof implantable in a cavity formed in the ulna and a second portion thereof, the first portion of said ulnar component defining a longitudinal axis thereof, the second portion of said ulnar component being rotatably connected to the first portion of said ulnar component about an axis normal to the longitudinal axis; and
a humeral component including a first portion thereof implantable in a cavity formed in the humerus and a second portion, the second portion of said humeral component being operabley connected to the second portion of said ulnar component.

2. The elbow prosthesis of claim 1:
wherein one of the first portion and the second portion of said ulnar component comprises a cylinder; and
wherein the other of the first portion and the second portion of said ulnar component defines an cylindrical opening therein adapted to receive said cylinder.

3. The elbow prosthesis of claim 1:
wherein the rotation of the second portion of said ulnar component with respect to the first portion of said ulnar component is limited to less than 360 degrees.

4. The elbow prosthesis of claim 3, wherein rotation of the second portion of said ulnar component with respect to the first portion of said ulnar component is limited to 10 to 60 degrees.

5. The elbow prosthesis of claim 1:
wherein one of the first portion of said ulnar component and the second portion of said ulnar component comprises a protrusion for restraining motion between the first portion of said ulnar component and the second portion of said ulnar component; and
wherein the other of the first portion of said ulnar component and the second portion of said ulnar component defines an void for cooperation with the protrusion.

6. The elbow prosthesis of claim 1, wherein the second portion of said humeral component is rotatably interlocked with the second portion of said ulnar component.

7. The elbow prosthesis of claim 1:
wherein the second portion of said ulnar component includes a contact surface for cooperation with the second portion of said humeral component;
wherein the second portion of said humeral component includes a contact surface for cooperation with the second portion of said ulnar component;
wherein the contact surface of the second portion of said humeral component is at least one of rollably or slidably connected to the contact surface of the second portion of said ulnar component; and
wherein the ulnar component may be freely separated from the humeral component in a direction normal to the contact surfaces.

8. The elbow prosthesis of claim 1:
wherein the first portion of said humeral component defines a longitudinal axis thereof; and
wherein the second portion of said humeral component is rotatably connected to the first portion of said humeral component about the longitudinal axis.

9. The elbow prosthesis of claim 1:
wherein one of the first portion and the second portion of said humeral component comprises an external taper; and
wherein the other of the first portion and the second portion of said humeral component defines an internal taper therein adapted to receive said external taper.

10. The elbow prosthesis of claim 1, further comprising a bearing positioned between the first portion of said humeral component and the second portion of said humeral component.

11. The elbow prosthesis of claim 1:
wherein one of the first and second portions of said ulnar component comprises an external taper; and
wherein the other of the first portion and the second portion of said ulnar component defines an internal taper therein adapted to receive said external taper.

12. An ulnar component for use with a humeral component to form an elbow prosthesis, said ulnar component comprising:
a first portion implantable in a cavity formed in the ulna, said first portion defining a longitudinal axis thereof; and
a second portion, said second portion being rotatably connected to said first portion about an axis normal to the longitudinal axis.

13. The ulnar component of claim 12:
wherein one of said first portion and said second portion comprises a cylinder; and
wherein the other of said first portion and said second portion defines an cylindrical opening therein adapted to receive said cylinder.

14. The ulnar component of claim 12:
wherein the rotation of said second portion with respect to said first portion is limited to less than 360 degrees.

15. The ulnar component of claim 12, wherein rotation of said second portion with respect to said first portion is limited to 10 to 60 degrees.

16. The ulnar component of claim 12:
wherein one of said first portion and said second portion comprises a protrusion for restraining motion between said first portion and said second portion; and
wherein the other of said first portion and said second portion defines an void for cooperation with said protrusion.

17. The ulnar component of claim 12:
wherein said second portion includes a contact surface adapted for cooperation with the humeral component;
wherein the contact surface of the second portion of said humeral component is adapted for at least one of rollably or slidably contact with ulnar component; and
wherein said second portion may be freely separated from the humeral component in a direction normal to the contact surface.

18. The ulnar component of claim 12, wherein said second portion is adapted to be rotatably interlocked with the humeral component.

19. The ulnar component of claim 18:
wherein second portion is removably, rotatably connected to said first portion; and
further comprising a third portion being removably, rotatably connected to said first portion about the longitudinal axis and adapted for at least one of rollably or slidably freely separable contact with humeral component.

20. A kit for use in performing total elbow arthroplasty, said kit comprising:
an ulnar stem component for implantation at least partially in the ulnar medullary canal, said ulnar stem component defining a longitudinal axis thereof;
a first ulnar hinge component attachable to said ulnar stem component for rotatable operation therewith about an axis normal to the longitudinal axis of said ulnar stem component;
a second ulnar hinge component attachable to said ulnar stem component for rotatable operation therewith about an axis normal to the longitudinal axis of said ulnar stem component said second ulnar hinge component being different in at least one of size and shape then said first ulnar hinge component;
a humeral stem component for implantation at least partially in the humeral medullary canal, said humeral stem component defining a longitudinal axis thereof; and
a humeral hinge component attachable to the humeral stem component and adapted for cooperation with said ulnar hinge component.
